SEQUENCE LISTING (37 sequences)

SEQ ID NO: 1 - DNA, 2811 bp, Corynebacterium glutamicum
  CDS (373)..(2022): pgi
aaaacccgaggggcgaaaattccaccctaacttttttgggatcccctttttccggggaattaattggtttgggtttcaatgggaaaacgggaaacaatgggccaaaggttcaaaaaccccaaaagggggccgggttcaaattcccaaaaaaaatggcaaaaaagggggggccaaaaccaagttggcccccaaaccaccggggcaacggcccacccacaaaggggttgggttaaaggaaggacgcccaaagtaagcccggaatggcccacgttcgaaaaagcaggccccaattaaacgcaccttaaatttgtcgtgtttcccactttgaacactcttcgatgcgcttggccacaaaagcaagctaacctgaagatgttatttaacgacaataaaggagttttcatggcggacatttcgaccacccaggtttggcaagacctgaccgatcattactcaaacttccaggcaaccactctgcgtgaacttttcaaggaagaaaaccgcgccgagaagtacaccttctccgcggctggcctccacgtcgacctgtcgaagaatctgcttgacgacgccaccctcaccaagctccttgcactgaccgaagaatctggccttcgcgaacgcattgacgcgatgtttgccggtgaacacctcaacaacaccgaagaccgcgctgtcctccacaccgcgctgcgccttcctgccgaagctgatctgtcagtagatggccaagatgttgctgctgatgtccacgaagttttgggacgcatgcgtgacttcgctactgcgctgcgctcaggcaactggttgggacacaccggccacacgatcaagaagatcgtcaacattggtatcggtggctctgacctcggaccagccatggctacgaaggctctgcgtgcatacgcgaccgctggtatctcagcagaattcgtctccaacgtcgacccagcagacctcgtttctgtgttggaagacctcgatgcagaatccacattgttcgtgatcgcttcgaaaactttcaccacccaggagacgctgtccaacgctcgtgcagctcgtgcttggctggtagagaagctcggtgaagaggctgtcgcgaagcacttcgtcgcagtgtccaccaatgctgaaaaggtcgcagagttcggtatcgacacggacaacatgttcggcttctgggactgggtcggaggtcgttactccgtggactccgcagttggtctttccctcatggcagtgatcggccctcgcgacttcatgcgtttcctcggtggattccacgcgatggatgaacacttccgcaccaccaagttcgaagagaacgttccaatcttgatggctctgctcggtgtctggtactccgatttctatggtgcagaaacccacgctgtcctaccttattccgaggatctcagccgttttgctgcttacctccagcagctgaccatggagaccaatggcaagtcagtccaccgcgacggctcccctgtttccactggcactggcgaaatttactggggtgagcctggcacaaatggccagcacgctttcttccagctgatccaccagggcactcgccttgttccagctgatttcattggtttcgctcgtccaaagcaggatcttcctgccggtgagcgcaccatgcatgaccttttgatgagcaacttcttcgcacagaccaaggttttggctttcggtaagaacgctgaagagatcgctgcggaaggtgtcgcacctgagctggtcaaccacaaggtcgtgccaggtaatcgcccaaccaccaccattttggcggaggaacttaccccttctattctcggtgcgttgatcgctttgtacgaacacaccgtgatggttcagggcgtgatttgggacatcaactccttcgaccaatggggtgttgaactgggcaaacagcaggcaaatgacctcgctccggctgtctctggtgaagaggatgttgactcgggagattcttccactgattcactgattaagtggtaccgcgcaaataggtagtcgcttgcttatagggtcaggggcgtgaagaatcctcgcctcatagcactggccgctatcatcctgacctcgttcaatctgcgaacagctattactgctttagctccgctggtttctgagattcgggatgatttaggggttagtgcttctcttattggtgtgttgggcatgatcccgactgctatgttcgcggttgctgcgtttgcgcttccgtcgttgaagaggaagttcactacttcccaactgttgatgtttgccatgctgttgactgctgccggtcagattattcgtgtcgctggacctgcttcgctgttgatggtcggtactgtgttcgcgatgtttgcgatcggagttaccaatgtgttgcttccgattgctgttagggagtattttccgcgtcacgtcggtggaatgtcgacaacttatctggtgtcgttccagattgttcaggcacttgctccgacgcttgccgtgccgatttctcagtgggctacacatgtggggttgaccggttggagggtgtcgctcggttcgtgggcgctgctggggttggttgcggcgatttcgtggattccgctgttgagtttgcagggtgccagggttgttgcggcgccgtcgaaggtttctcttcctgtgtggaagtcttcggttggtgtggggctcgggttgatgtttgggtttacttcgtttgcgacgtatatcctcatgggttttatgccgcagatggtaggtgatccaaagaattcaaaaagcttctcgagagtacttctagagcggccgcgggcc

SEQ ID NO: 2 - protein, 550 aa, Corynebacterium glutamicum
MLFNDNKGVFMADISTTQVWQDLTDHYSNFQATTLRELFKEENRAEKYTFSAAGLHVDLSKNLLDDATLTKLLALTEESGLRERIDAMFAGEHLNNTEDRAVLHTALRLPAEADLSVDGQDVAADVHEVLGRMRDFATALRSGNWLGHTGHTIKKIVNIGIGGSDLGPAMATKALRAYATAGISAEFVSNVDPADLVSVLEDLDAESTLFVIASKTFTTQETLSNARAARAWLVEKLGEEAVAKHFVAVSTNAEKVAEFGIDTDNMFGFWDWVGGRYSVDSAVGLSLMAVIGPRDFMRFLGGFHAMDEHFRTTKFEENVPILMALLGVWYSDFYGAETHAVLPYSEDLSRFAAYLQQLTMETNGKSVHRDGSPVSTGTGEIYWGEPGTNGQHAFFQLIHQGTRLVPADFIGFARPKQDLPAGERTMHDLLMSNFFAQTKVLAFGKNAEEIAAEGVAPELVNHKVVPGNRPTTTILAEELTPSILGALIALYEHTVMVQGVIWDINSFDQWGVELGKQQANDLAPAVSGEEDVDSGDSSTDSLIKWYRANR

SEQ ID NO: 3 - DNA, 462 bp, Corynebacterium glutamicum
atggagaccaatggcaagtcagtccaccgcgacggctcccctgtttccactggcactggcgaaatttactggggtgagcctggcacaaatggccagcacgctttcttccagctgatccaccagggcactcgccttgttccagctgatttcattggtttcgctcgtccaaagcaggatcttcctgccggtgagcgcaccatgcatgaccttttgatgagcaacttcttcgcacagaccaaggttttggctttcggtaagaacgctgaagagatcgctgcggaaggtgtcgcacctgagctggtcaaccacaaggtcgtgccaggtaatcgcccaaccaccaccattttggcggaggaacttaccccttctattctcggtgcgttgatcgctttgtacgaacacaccgtgatggttcagggcgtgatttgggacatcaactccttcgaccaatggggcgtggaa

SEQ ID NO: 4 - DNA, 2160 bp, Corynebacterium glutamicum
  CDS (327)..(2063): poxB
ttagaggcgattctgtgaggtcactttttgtggggtcggggtctaaatttggccagttttcgaggcgaccagacaggcgtgcccacgatgtttaaataggcgatcggtgggcatctgtgtttggtttcgacgggctgaaaccaaaccagactgcccagcaacgacggaaatcccaaaagtgggcatccctgtttggtaccgagtacccacccgggcctgaaactccctggcaggcgggcgaagcgtggcaacaactggaatttaagagcacaattgaagtcgcaccaagttaggcaacacaatagccataacgttgaggagttcagatggcacacagctacgcagaacaattaattgacactttggaagctcaaggtgtgaagcgaatttatggtttggtgggtgacagccttaatccgatcgtggatgctgtccgccaatcagatattgagtgggtgcacgttcgaaatgaggaagcggcggcgtttgcagccggtgcggaatcgttgatcactggggagctggcagtatgtgctgcttcttgtggtcctggaaacacacacctgattcagggtctttatgattcgcatcgaaatggtgcgaaggtgttggccatcgctagccatattccgagtgcccagattggttcgacgttcttccaggaaacgcatccggagattttgtttaaggaatgctctggttactgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgcgattggtgcgcaaagtgttgatcgaaaccgccaggtgatcgcgatgtgtggcgatggtggtttgggcatgctgctgggtgagcttctgaccgttaagctgcaccaacttccgctgaaggctgtggtgtttaacaacagttctttgggcatggtgaagttggagatgctcgtggagggacagccagaatttggtactgaccatgaggaagtgaatttcgcagagattgcggcggctgcgggtatcaaatcggtacgcatcaccgatccgaagaaagttcgcgagcagctagctgaggcattggcatatcctggacctgtactgatcgatatcgtcacggatcctaatgcgctgtcgatcccaccaaccatcacgtgggaacaggtcatgggattcagcaaggcggccacccgaaccgtctttggtggaggagtaggagcgatgatcgatctggcccgttcgaacataaggaatattcctactccatgatgattgatacacctgctgttctcattgaccgcgagcgcttaactgccaacatttccaggatggcagctcacgccggtgcccatgagattgccct

SEQ ID NO: 5 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 6 - DNA, 875 bp, Corynebacterium glutamicum
tgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgc

SEQ ID NO: 7 - DNA, 2260 bp, Brevibacterium flavum MJ-233
  CDS (629)..(2080): Glucose-6-Phosphate Dehydrogenase (EC 1.1.1.49); JP-A-09-22461
gatccgatgaggctttggctctgcgtggcaaggcaggcgttgccaacgctcagcgcgcttacgctgtgtacaaggagcttttcgacgccgccgagctgcctgtaaggcgccaacactcagcgcccactgtgggcatccaccggcgtgaagaaccctgcgtacgctgcaactctttacgtttccgagctggctggtccaaacaccgtcaacaccatgccagaaggcaccatcgacgctgttctggaactgggcaacctgcacggtgacaacctgtccaactccgcggcagaagctgacgctgtgttctcccagcttgaggctctgggcgttgacttggcagatgtcttccaggtcctggagaccgaggccgtggacaagttcgttgcttcttggagcgaactgcttgagtccatggaagctcgcctgaagtagaatcagcacgctgcatcagtaacggcgacatgaaatcgaattagttcgatcttatgtggccgttacacatctttcattaaagaaaggatcgtgacgcttaccatcgtgagcacaaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcgcagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgccttatcttggatgcgctgttggatgaatccagccttttccctaccaacgaggaagtggaactgagctggaagattctggatccaattcttgaagcatgggatgccgatggagaaccagaggattacccagcaggtacgtggggtccaaagagcgctgatgaaatgctttcccgcaacggtcacacctggcgcaggccataatttaggggcaaaaaatgatctttgaacttccggataccaccacccagcaaatttccaagaccctaactcgactgcgtgaatcgggcacccaggtcaccaccggccgagtgctcaccctcatcgtggtcactgactccgaaagcgatgtcgctgcagttaccgagtccaccaatgaag

SEQ ID NO: 8 - protein, 484 aa, Brevibacterium flavum MJ-233
MVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFAAVCHQLERSGMAESTEEAWRRVIIEKPFGHNLESAHELNQLVNAVFPESSVFRIDHYLGKETVQNILALRFANQLFEPLWNSNYVDHVQITMAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 9 - DNA, 2259 bp, Corynebacterium glutamicum
  CDS (538)..(2079): Zwf-Protein
gatccgatgaggctttggctctgcgtggcaaggcaggcgttgccaacgctcagcgcgcttacgctgtgtacaaggagcttttcgacgccgccgagctgcctgtaaggcgccaacactcagcgcccactgtgggcatccaccggcgtgaagaaccctgcgtacgctgcaactctttacgtttccgagctggctggtccaaacaccgtcaacaccatgccagaaggcaccatcgacgctgttctggaactgggcaacctgcacggtgacaacctgtccaactccgcggcagaagctgacgctgtgttctcccagcttgaggctctgggcgttgacttggcagatgtcttccaggtcctggagaccgaggccgtggacaagttcgttgcttcttggagcgaactgcttgagtccatggaagctcgcctgaagtagaatcagcacgctgcatcagtaacggcgacatgaaatcgaattagttcgatcttatgtggccgttacacatctttcattaaagaaaggatcgtgacgcttaccatcgtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcgcagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgccttatcttggatgcgctgttggatgaatccagccttttccctaccaacgaggaagtggaactgagctggaagattctggatccaattcttgaagcatgggatgccgatggagaaccagaggattacccagcaggtacgtggggtccaaagagcgctgatgaaatgctttcccgcaacggtcacacctggcgcaggccataatttaggggcaaaaaatgatctttgaacttccggataccaccacccagcaaatttccaagaccctaactcgactgcgtgaatcgggcacccaggtcaccaccggccgagtgctcaccctcatcgtggtcactgactccgaaagcgatgtcgctgcagttaccgagtccaccaatgaag

SEQ ID NO: 10 - protein, 514 aa, Corynebacterium glutamicum
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFAAVCHQLERSGMAESTEEAWRRVIIEKPFGHNLESAHELNQLVNAVFPESSVFRIDHYLGKETVQNILALRFANQLFEPLWNSNYVDHVQITMAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 11 - DNA, 20 bp, Artificial Sequence
  Primer zwf-forward
tcgacgcggttctggagcag

SEQ ID NO: 12 - DNA, 21 bp, Artificial Sequence
  Primer zwf reverse
ctaaattatggcctgcgccag

SEQ ID NO: 13 - DNA, 22 bp, Artificial Sequence
  Universal forward Primer
gtaatacgactcactatagggc

SEQ ID NO: 14 - DNA, 18 bp, Artificial Sequence
  M13 reverse primer
ytccacgccccaytgrtc

SEQ ID NO: 15 - DNA, 18 bp, Artificial Sequence
  Internal Primer 1
ggaaacaggggagccgtc

SEQ ID NO: 16 - DNA, 18 bp, Artificial Sequence
  Internal Primer 2
tgctgagataccagcggt

SEQ ID NO: 17 - DNA, 17 bp, Artificial Sequence
  fwd. primer
atggarwccaaygghaa

SEQ ID NO: 18 - DNA, 18 bp, Artificial Sequence
  rev. primer
ytccacgccccaytgrtc

SEQ ID NO: 19 - DNA, 20 bp, Artificial Sequence
  Primer poxBint1
tgcgagatggtgaatggtgg

SEQ ID NO: 20 - DNA, 20 bp, Artificial Sequence
  Primer poxBint2
gcatgaggcaacgcattagc

SEQ ID NO: 21 - DNA, 1857 bp, Corynebacterium glutamicum
  CDS (308)..(1849)
tcgacgcggttctggagcagggcaacctgcacggtgacaccctgtccaactccgcggcagaagctgacgctgtgttctcccagcttgaggctctgggcgttgacttggcagatgtcttccaggtcctggagaccgagggtgtggacaagttcgttgcttcttggagcgaactgcttgagtccatggaagctcgcctgaagtagaatcagcacgctgcatcagtaacggcgacatgaaatcgaattagttcgatcttatgtggccgttacacatctttcattaaagaaaggatcgtgacactaccatcgtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatgactgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttagatgaatccagcctcttccctaccaacgaggaagtggaactgagctggaagattctggatccaattcttgaagcatgggatgccgatggagaaccagaggattacccagcgggtacgtggggtccaaagagcgctgatgaaatgctttcccgcaacggtcacacctggcgcaggccataatttag

SEQ ID NO: 22 - protein, 514 aa, Corynebacterium glutamicum
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFTAVCHQLERSGMAESTEEAWRRVIIEKPFGHNLESAHELNQLVNAVFPESSVFRIDHYLGKETVQNILALRFANQLFEPLWNSNYVDHVQITMTEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 23 - DNA, 756 bp, Corynebacterium glutamicum
  misc_feature (1)..(756): Internal segment of the coding sequence of the zwf(A243T) allele
agaatcttctgtgttccgcatcgaccactatttgggcaaggaaacagttcaaaacatcctggctctgcgttttgctaaccagctgtttgagccactgtggaactccaactacgttgaccacgtccagatcaccatgactgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcct

SEQ ID NO: 24 - DNA, 28 bp, Artificial Sequence
  Primer zwf_XL-A1
gatctagaagctcgcctgaagtagaatc

SEQ ID NO: 25 - DNA, 28 bp, Artificial Sequence
  Primer zwf_XL-E1
gatctagagattcacgcagtcgagttag

SEQ ID NO: 26 - DNA, 1763 bp, Corynebacterium glutamicum
  PCR product (1)..(1763)
gatctagaagctcgcctgaagtagaatcagcacgctgcatcagtaacggcgacatgaaatcgaattagttcgatcttatgtggccgttacacatctttcattaaagaaaggatcgtgacactaccatcgtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatattgacttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttagatgaatccagcctcttccctaccaacgaggaagtggaactgagctggaagattctggatccaattcttgaagcatgggatgccgatggagaaccagaggattacccagcgggtacgtggggtccaaagagcgctgatgaaatgctttcccgcaacggtcacacctggcgcaggccataatttaggggcaaaaaatgatctttgaacttccggataccaccacccagcaaatttccaagaccctaactcgactgcgtgaatctctagatc

SEQ ID NO: 27 - DNA, 20 bp, Artificial Sequence
  Primer zf_1
ggcttactacctgtccattc

SEQ ID NO: 28 - DNA, 1545 bp, Artificial Sequence
  Obtained by in-vitro mutagenesis
gtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtatgcgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttagatgaatccagcctcttccctaccaacgaggaagtggaactgagctggaagattctggatccaattcttgaagcatgggatgccgatggagaaccagaggattacccagcgggtacgtggggtccaaagagcgctgatgaaatgctttcccgcaacggtcacacctggcgcaggccataa

SEQ ID NO: 29 - protein, 514 aa, Artificial Sequence
  Obtained by in-vitro mutagenesis
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFTAVCHQLERSGMAESTEEAWRRVIIEKPFGHNLESAHELNQLVNAVFPESSVFRIDHYLGKETVQNILALRFANQLFEPLWNSNYVDHVQITMAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGMRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 30 - DNA, 1545 bp, Artificial Sequence
  Obtained by in-vitro mutagenesis
gtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgcaactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttagatgaatccagcctcttccctaccaacgaggaagtggaactgagctggaagattctggatccaattcttgaagcatgggatgccgatggagaaccagaggattacccagcgggtacgtggggtccaaagagcgctgatgaaatgctttcccgcaacggtcacacctggcgcaggccataa

SEQ ID NO: 31 - protein, 514 aa, Artificial Sequence
  Obtained by in-vitro mutagenesis
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFTAVCHQLERSGMAESTEEAWRRVIIEKPFGHNLESAHELNQLVNAVFPESSVFRIDHYLGKETVQNILALRFANQLFEPLWNSNYVDHVQITMAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRATEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 32 - DNA, 1545 bp, Artificial Sequence
  Obtained by in vitro mutagenesis
gtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcacccttgctgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttagatgaatccagcctcttccctaccaacgaggaagtggaactgagctggaagattctggatccaattcttgaagcatgggatgccgatggagaaccagaggattacccagcgggtacgtggggtccaaagagcgctgatgaaatgctttcccgcaacggtcacacctggcgcaggccataa

SEQ ID NO: 33 - protein, 514 aa, Artificial Sequence
  Obtained by in vitro mutagenesis
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFTAVCHQLERSGMAESTEEAWRRVIIEKPFGHNLESAHELNQLVNAVFPESSVFRIDHYLGKETVQNILALRFANQLFEPLWNSNYVDHVQITLAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 34 - DNA, 1545 bp, Artificial Sequence
  Obtained by in-vitro mutagenesis
gtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcacctcagctgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttagatgaatccagcctcttccctaccaacgaggaagtggaactgagctggaagattctggatccaattcttgaagcatgggatgccgatggagaaccagaggattacccagcgggtacgtggggtccaaagagcgctgatgaaatgctttcccgcaacggtcacacctggcgcaggccataa

SEQ ID NO: 35 - protein, 514 aa, Artificial Sequence
  Obtained by in-vitro mutagenesis
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFTAVCHQLERSGMAESTEEAWRRVIIEKPFGHNLESAHELNQLVNAVFPESSVFRIDHYLGKETVQNILALRFANQLFEPLWNSNYVDHVQITSAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 36 - DNA, 1545 bp, Artificial Sequence
  Obtained by in-vitro mutagenesis
gtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaatcaattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttagatgaatccagcctcttccctaccaacgaggaagtggaactgagctggaagattctggatccaattcttgaagcatgggatgccgatggagaaccagaggattacccagcgggtacgtggggtccaaagagcgctgatgaaatgctttcccgcaacggtcacacctggcgcaggccataa

SEQ ID NO: 37 - protein, 514 aa, Artificial Sequence
  Obtained by in-vitro mutagenesis
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFTAVCHQLERSGMAESTEEAWRRVIIEKPFGHNLESAHELNQLVNAVFPESSVFRIDHYLGKETVQNILALRFANQLFEPLWNSNYVDHVQITMAESIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

(19) United States
(12) Patent Application Publication
(10) Pub. No.: US 2003/0175911 A1
(43) Pub. Date: Sep. 18, 2003

(54) Process for the preparation of L-amino acids with amplification of the zwf gene

(76) Inventors: Stephen Hans, Osnabruek (DE); Brigitte Bathe, Salzkotten (DE); Alexander Reth, Bielefeld (DE); Georg Thierbach, Bielefeld (DE); Caroline Kreutzer, Melle (DE); Bettina Mockel, Dusseldorf (DE)

Correspondence Address: Michael A. Sanzo, Fitch, Even, Tabin & Flannery, Suite 401L, 1801 K Street, N.W., Washington, DC, 20006-1201, US

(21) Appl. No.: 10/336,049

(22) Filed: Jan. 3, 2003

Related U.S. Application Data
(63) Continuation-in-part of application No. 10/091,342, filed on Mar. 6, 2002.
Application No. 10/091,342 is a continuation-in-part of application No. 09/531,269, filed on Mar. 20, 2000, now abandoned.

(51) Int. Cl. C12P 13/08; C12N 1/21

(52) U.S. Cl. 435/115; 435/252.3

(57) ABSTRACT

The invention relates to a process for the preparation of L-amino acids by the fermentation of coryneform bacteria. The process involves: fermenting an L-amino acid-producing bacteria in which at least the zwf gene is amplified; concentrating the L-amino acid in the medium or in the cells of the bacteria; and isolating the L-amino acid produced.

### CROSS REFERENCE TO RELATED APPLICATIONS

The present application is a continuation-in-part of U.S. Ser. No. 10/091,342, filed on Mar. 6, 2002, which is a continuation-in-part of U.S. Ser. No. 09/531,269, filed Mar. 20, 2000.

### FIELD OF THE INVENTION

The invention relates to a process for the preparation of L-amino acids, particularly L-lysine, L-threonine and L-tryptophan, using coryneform bacteria in which at least the Zwischenferment protein encoded by the zwf gene is amplified.

### BACKGROUND OF THE INVENTION

L-Amino acids are used in animal nutrition, in human medicine and in the pharmaceuticals industry. One effective manner of producing amino acids for these purposes is by the fermentation of strains of coryneform bacteria and, in particular, Corynebacterium glutamicum. Because of its great importance, improvements are constantly being made in this process. Such improvements may relate to fermentation procedures (e.g., the stirring of preparations or supply of oxygen) or to the composition of the nutrient media (e.g., the sugar concentration present during fermentation). Alternatively, improvements may relate to the methods by which product is purified or to the intrinsic synthetic properties of the microorganism itself.

Methods of mutagenesis and selection have been used to increase the amount of amino acid produced by microorganisms. Strains which are resistant to antimetabolites (e.g., the threonine analogue α-amino-β-hydroxyvaleric acid (AHV) or the lysine analogue S-(2-aminoethyl)-L-cystein (AEC)) or that are auxotrophic for metabolites of regulatory importance and produce L-amino acids (e.g., threonine or lysine) may be obtained in this manner. In addition, recombinant DNA techniques have been used to improve the production characteristics of Corynebacterium glutamicum strains.

### OBJECT OF THE INVENTION

The object of the present invention is to provide improved procedures for the fermentative preparation of L-amino acids by coryneform bacteria.

### SUMMARY OF THE INVENTION

The present invention provides a process for the preparation of L-amino acids, particularly L-lysine, L-threonine, L-isoleucine and L-tryptophan, using coryneform bacteria in which the Zwischenferment protein (Zwf protein) encoded by the nucleotide sequence of the zwf gene is amplified, in particular over-expressed. The abbreviation “zwf” is a mnemonic for “Zwischenferment” (Jeffrey H. Miller: A Short Course In Bacterial Genetics, Cold Spring Harbor Laboratory Press, USA, 1992) and is also referred to as glucose 6-phosphate dehydrogenase. This enzyme catalyzes the oxidation of glucose-6-phosphate to 6-phosphogluconolactone by concomitant reduction of NADP to NADPH. Its activity is inhibited by NADPH and various other metabolites (Sugimoto, et al., Agri. Biol. Chem. 51(1):101-108 (1987)).

### BRIEF DESCRIPTION OF THE FIGURES

FIG. 1: Map of the plasmid pEC-T18mob2. In this and all other figures, the base pair numbers stated are approximate values obtained in the context of reproducibility. The meaning of the abbreviations for the various restriction enzymes (e.g. BamHI, EcoRI etc.) are known from the prior art and are summarized, for example, by Kessler et al. (Gene 47:1-153 (1986)) or by Roberts et al. (Nucl. Ac. Res. 27:312-313 (1999)). The abbreviations used in this figure and in FIG. 2 have the following meaning:

| | |
|---|---|
| Tet: | Resistance gene for tetracycline |
| oriV: | Plasmid-coded replication origin of E. coli |
| RP4mob: | mob region for mobilizing the plasmid |
| rep: | Plasmid-coded replication origin from C. glutamicum |
| | plasmid pGA1 |
| per: | Gene for controlling the number of copies from pGA1 |
| lacZ-alpha: | lacZα gene fragment (N-terminus) of the β-galactosidase |
| | gene |
| lacZalpha′: | 5′-Terminus of the lacZα gene fragment |
| ′lacZalpha: | 3′-Terminus of the lacZα gene fragment |

FIG. 2: Map of the plasmid pEC-T18mob2zwf.

FIG. 3: Map of the plasmid pAMC1. The abbreviations used here and in

FIG. 4 have the following meaning:

| | |
|---|---|
| Neo r: | Neomycin/kanamycin resistance |
| ColE1 ori: | Replication origin of the plasmid ColE1 |
| CMV: | Cytomegalovirus promoter |
| lacP: | Lactose promoter |
| pgi: | Phosphoglucose isomerase gene |
| lacZ: | Part of the β-galactosidase gene |
| SV40 3′ splice | 3′ splice site of Simian virus 40 |
| SV40 polyA: | Polyadenylation site of Simian virus 40 |
| f1(-)ori: | Replication origin of the filamentous phage f1 |
| SV40 ori: | Replication origin of Simian virus 40 |
| kan r: | Kanamycin resistance |
| pgi insert: | Internal fragment of the pgi gene |
| ori: | Replication origin of the plasmid pBGS8 |

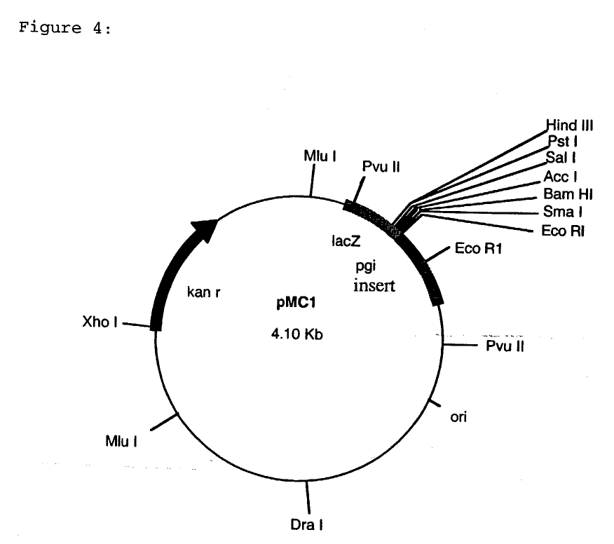

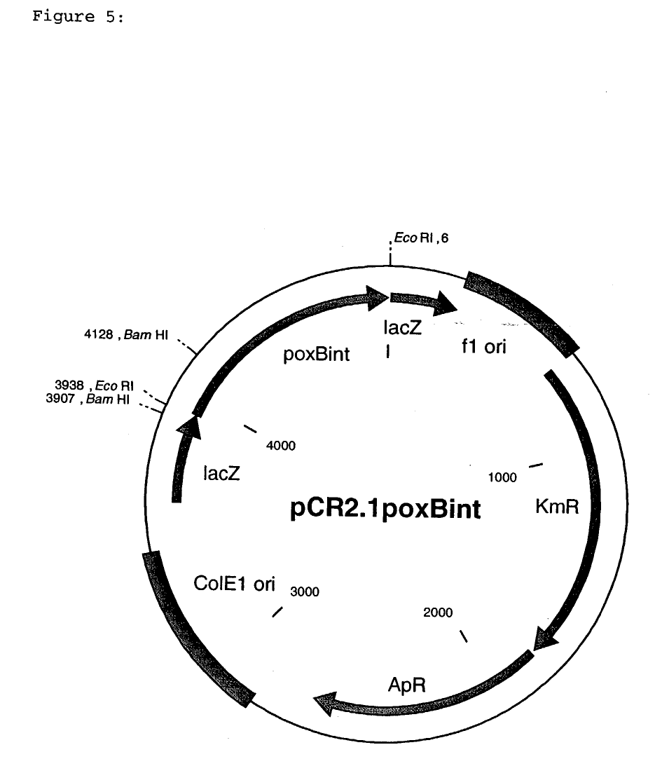

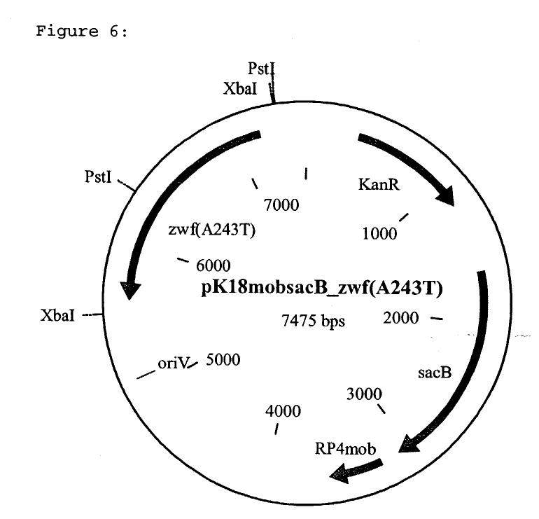

FIG. 4: Map of the plasmid pMC1.

FIG. 5: Map of the plasmid pCR2.1poxBint. The abbreviations used in the figure have the following meaning:

| | |
|---|---|
| ColE1 ori: | Replication origin of the plasmid ColE1 |
| lacZ: | Cloning relict of the lacZα gene fragment |
| f1 ori: | Replication origin of phage f1 |
| KmR: | Kanamycin resistance |
| ApR: | Ampicillin resistance |
| poxBint: | Internal fragment of the poxB gene |

FIG. 6: Map of the plasmid pK18mobsacB_zwf(A243T). The abbreviations used in the figure have the following meaning:

| | |
|---|---|
| RP4mob: | mob region with the replication origin for the transfer (oriT) |
| KanR: | Kanamycin resistance gene |
| oriV: | Replication origin V |
| zwf(A243T): | zwf(A243T) allele |
| sacB: | sacB gene. |

### DETAILED DESCRIPTION OF THE INVENTION

The strains of bacteria employed in the present invention preferably already produce L-amino acids before amplification of the zwf gene. The term “amplification” in this connection describes the increase in the intracellular activity of one or more enzymes or proteins in a microorganism which are coded by the corresponding DNA. Amplification may be achieved, for example, by increasing the number of copies of the gene or genes, by using a potent promoter to increase expression or by using a gene or allele which codes for a corresponding protein having high enzymatic activity. Also, several different methods of amplification may, optionally, be combined. As the result of amplification measures, in particular over-expression, the activity or concentration of the corresponding enzyme or protein can be increased by at least 10%, 25%, 50%, 75%, 100%, 150%, 200%, 300%, 400% or 500%, up to a maximum of 1000% or 2000%, relative to that of the wild-type enzyme or protein or the activity or concentration of the enzyme or protein in the starting microorganism.

The microorganisms of the present invention can prepare L-amino acids from glucose, sucrose, lactose, fructose, maltose, molasses, starch, cellulose or from glycerol and ethanol. They are representatives of coryneform bacteria, and, in particular, of the genus Corynebacterium. Of the genus Corynebacterium, the most preferred species is Corynebacterium glutamicum, which is known among specialists for its excellent ability to produce L-amino acids. Suitable wild-type strains of the genus Corynebacterium, in particular of the species Corynebacterium glutamicum, include:

Corynebacterium glutamicum ATCC13032;

Corynebacterium acetoglutamicum ATCC15806;

Corynebacterium acetoacidophilum ATCC13870;

Corynebacterium thermoaminogenes FERM BP-1539;

Brevibacterium flavum ATCC14067;

Brevibacterium lactoferrnentum ATCC 13869;

Brevibacterium divaricatum ATCC 14020;

and L-amino acid-producing mutants prepared therefrom. Suitable mutant strains include:

A. The L-threonine-producing strains:

Corynebacterium glutamicum ATCC21649;

Brevibacterium flavum BB69;

Brevibacterium flavum DSM5399;

Brevibacterium lactofermentum FERM-BP 269;

Brevibacterium lactofermentum TBB-10;

B. The L-isoleucine-producing strains:

Corynebacterium glutamicum ATCC 14309;

Corynebacterium glutamicum ATCC 14310;

Corynebacterium glutamicum ATCC 14311;

Corynebacterium glutamicum ATCC 15168;

Corynebacterium ammoniagenes ATCC 6871;

C. The L-tryptophan-producing strains:

Corynebacterium glutamicum ATCC21850; and

Corynebacterium glutamicum KY9218(pKW9901);

D. The L-lysine-producing strains:

Corynebacterium glutamicum FERM-P 1709;

Brevibacterium flavum FERM-P 1708;

Brevibacterium lactofermentum FERM-P 1712;

Corynebacterium glutamicum FERM-P 6463;

Corynebacterium glutamicum FERM-P 6464;

Corynebacterium glutamicum ATCC1 3032;

Corynebacterium glutamicum DM58-1; and

Corynebacterium glutamicum DSM12866.

It has been found that coryneform bacteria produce L-amino acids, particularly L-lysine, L-threonine and L-tryptophan, in an improved manner after over-expression of the zwf gene which codes for the Zwf protein or polypeptide. JP-A-09224661 discloses the nucleotide sequence of the zwf gene of Brevibacterium flavum MJ-223 (FERM BP-1497) and refers to the protein encoded by the zwf-gene as glucose 6-phosphate dehydrogenase. The sequence information disclosed in JP-A-09224661 is shown in SEQ ID NOs: 7 and 8. JP-A-09224661 suggests that the N-terminal amino acid sequence of the Zwf polypeptide is Met Val Ile Phe Gly Val Thr Gly Asp Leu Ala Arg Lys Lys Leu (SEQ ID NO: 8). However, an alternative form of the gene and enzyme have now been discovered which, instead, have the following N-terminal amino acid sequence: Met Ser Thr Asn Thr Thr Pro Ser Ser Trp Thr Asn Pro Leu Arg Asp (SEQ ID NO: 10). The nucleotide sequence of the corresponding zwf gene includes the coding sequence is shown in SEQ ID NO: 9. The methionine residue in the N-position can be split off in the due to post-translational modification, and Ser Thr Asn Thr Thr Pro Ser Ser Trp Thr Asn Pro Leu Arg Asp is then obtained as the N-terminal amino acid sequence. Accordingly, this invention provides the nucleotide sequence of a novel zwf gene from a coryneform bacterium shown in SEQ ID NO: 9, nucleotides 538 to 2079.

Genes encoding Zwf proteins from Gram-negative bacteria e.g., Escherichia coli or other Gram-positive bacteria, e.g., Streptomyces or Bacillus, may optionally be used to increase Zwf expression in Corynebacterium. Alleles of the zwf gene which result from the degeneracy of the genetic code or due to sense mutations of neutral function can also be used. However, the use of endogenous genes, in particular endogenous genes from coryneform bacteria, is preferred. “Endogenous genes” or “endogenous nucleotide sequences” refers to genes or nucleotide sequences which are available in the population of a species.

To achieve an amplification (e.g., over-expression), the number of copies of the corresponding genes may be increased, or the promoter, regulation region or ribosome binding site upstream of the structural gene may be mutated. Expression cassettes which are incorporated upstream of the structural gene may be used for this purpose. Using inducible promoters, it is additionally possible to increase the expression of one or more amino acids during the course of a fermentative procedure. Expression may also be improved by measures that prolong the life of m-RNA and enzymatic activity can be increased by preventing the degradation of the protein. Genes or gene constructs may be delivered to bacteria in plasmids with a varying number of copies, or a gene may be integrated into the bacterial genome and then amplified.

Another approach to over-expressing genes is by changing the composition of the bacterial growth medium and the culture procedure. Instructions in this context can be found, inter alia, in Martin et al (Bio/Technology 5:137-146 (1987)), Guerrero et al. (Gene 138:35-41 (1994)), Tsuchiya, et al. (Bio/Technology 6:428-430 (1988)), Eikmanns et al. (Gene 102:93-98 (1991)), European Patent Specification EPS 0 472 869, U.S. Pat. No. 4,601,893, Schwarzer et al. (Bio/Technology 9:84-87 (1991)), Reinscheid, et al. (Appl. Envir. Microbiol. 60:126-132 (1994)), LaBarre et al. (J. Bacteriol. 175:1001-1007 (1993)), patent application WO 96/15246, Malumbres, et al. (Gene 134:15-24 (1993)), Japanese laid-open specification JP-A-10-229891, Jensen, et al., (Biotech. Bioeng. 58:191-195 (1998)) and in textbooks of genetics and molecular biology.

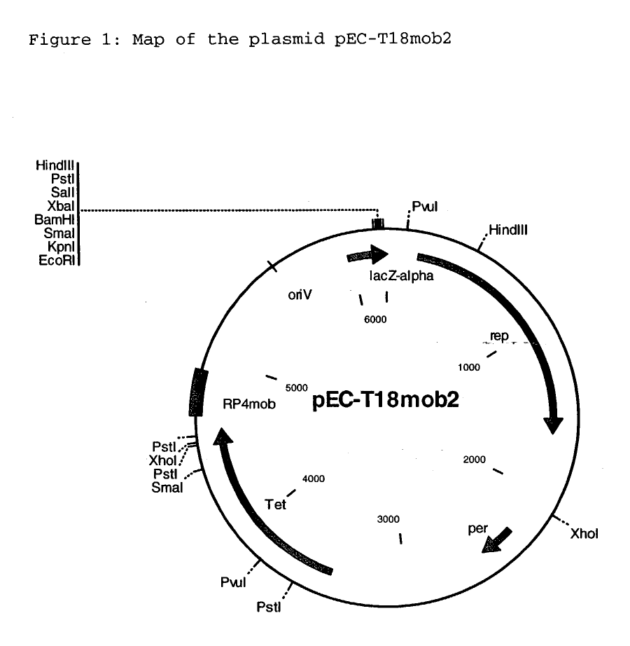

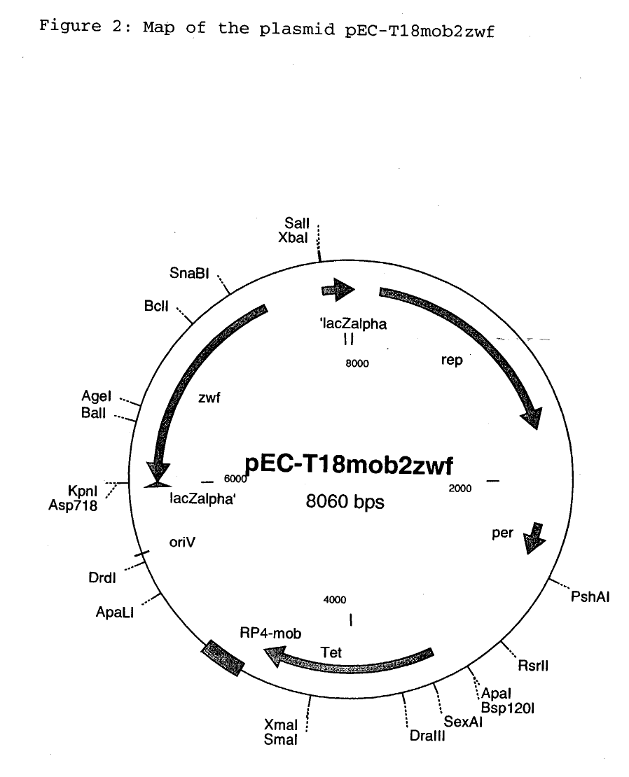

By way of example, the Zwf protein was over-expressed with the aid of the E. coli—C. glutamicum shuttle vector pEC-T18mob2 shown in FIG. 1. After incorporation of the zwf gene into the KpnI/SalI cleavage site of pEC-T18mob2, the plasmid pEC-T18mob2zwf, shown in FIG. 2, was formed. Other plasmid vectors which are capable of replication in C. glutamicum, e.g. pEKE×1 (Eikmanns et al., Gene 102:93-98 (1991)) or pZ8-1 (EP-B-0 375 889), can be used in the same way.

In a further aspect of the invention, it has been found that amino acid exchanges in the section between position 369 and 373 and/or position 241 and 246 of the amino acid sequence of the zwf gene product, as shown in SEQ ID NO: 10, amplify its glucose 6-phosphate dehydrogenase activity. This appears to be due to a decrease in the susceptibility of the enzyme to inhibition by NADPH (nicotinamide adenine dinucleotide phosphate, reduced form) resulting in an improvement in the production of amino acids, especially lysine, by coryneform bacteria. The methionine residue in the N-terminal position can be removed during post translational modification by a methionine aminopeptidase of the host. Accordingly, the invention provides Zwf proteins comprising the amino acid sequence of SEQ ID NO: 10, wherein at least one or more of the amino acids at positions 369 to 373 and/or one or more of the amino acids at positions 241 to 246 is (are) exchanged by another proteinogenic amino acid. In addition, the invention provides isolated polynucleotides encoding Zwf proteins containing these mutations.

Among the preferred exchanges within the amino acid sequence of the Zwf protein are: exchange of L-arginine at position 370 of SEQ ID NO: 10 for any other proteinogenic amino acid, e.g., L-methionine; exchange of L-valine at position 372 of SEQ ID NO: 10 for any other proteinogenic amino acid, e.g., L-alanine; exchange of L-methionine at position 242 of SEQ ID NO: 10 for any other proteinogenic amino acid e.g. L-leucine or L-serine; exchange of L-alanine at position 243 of SEQ ID NO: 10 for any other proteinogenic amino acid, e.g. L-threonine; exchange of L-glutamic acid at position 244 of SEQ ID NO: 10 for any other proteinogenic amino acid; and exchange of L-aspartic acid at position 245 of SEQ ID NO: 10 for any other proteinogenic amino acid, e.g. L-serine. The most preferred of these exchanges is L-alanine at position 243 (see SEQ ID NO: 10) for L-threonine as shown in SEQ ID NO: 22. This protein is also referred to as Zwf(A243T) protein and the allele encoding said protein is referred to as zwf(A243T; see also SEQ ID NO: 21). Other changes that may be made include the following:

L-arginine at position 370 (see SEQ ID NO: 10) can be exchanged for L-methionine as shown in SEQ ID NO: 29. This protein is also referred to as Zwf(R370M) and the allele encoding the protein is referred to as zwf(R370M) See also SEQ ID NO: 28.

L-valine at position 372 (see SEQ ID NO: 10) can be exchanged for L-alanine as shown in SEQ ID NO: 3 1. This protein is also referred to as Zwf(V372A) and the allele encoding the protein is referred to as zwf(V372A). See also SEQ ID NO: 30.

L-methionine at position 242 (see SEQ ID NO: 10) can be exchanged for L-leucine as shown in SEQ ID NO: 33. This protein is also referred to as Zwf(M242L) and the allele encoding the protein is referred to as zwf(M242L). See also SEQ ID NO: 32.

L-methionine at position 242 (see SEQ ID NO: 10) can be exchanged for L-serine as shown in SEQ ID NO: 35. This protein is also referred to as Zwf(M242S) and the allele encoding the protein is referred to as zwf(M242S). See also SEQ ID NO: 34.

L-aspartic acid in position 245 (see SEQ ID NO: 10) can be exchanged for L-serine as shown in SEQ ID NO: 37. This protein is also referred to as Zwf(D245S) and the allele encoding said protein is referred to as zwf(D245S). See also SEQ ID NO: 36.

The Zwf proteins according to the invention may contain further substitutions, deletions or insertions of one or more amino acids which do not substantially change the enzymatic properties of the Zwf protein variants described. For example, a change of enzymatic activity in the presence of the inhibitor NADPH of less than approximately 2.5 to 3.5% or 2.5 to 4.5% can be regarded as not substantially different. In the case of other parameters like the Michaelis constant (K_{M}), maximal rate (Vₘₐₓ) or other binding constants, differences less than approximately 5, 10, 25, 50, 100, 150 or 200% or even larger differences might be regarded as not substantially different.

Accordingly, the Zwf(A243T) protein comprises at least an amino acid sequence selected from the group consisting of Thr Met Thr Glu Asp Ile corresponding to the amino acids at positions 241 to 246 of SEQ ID NO: 22, the amino acid sequence corresponding to the amino acids at positions 235 to 250 of SEQ ID NO: 22, the amino acid sequence corresponding to the amino acids at positions 225 to 260 of SEQ ID NO: 22 and the amino acid sequence corresponding to the amino acids at positions 210 to 270 of SEQ ID NO: 22. Similarly, the Zwf protein variants Zwf(M242L), Zwf(M242S) and Zwf(D245) comprise at least an amino acid sequence selected from the group consisting of the amino acid sequence of the amino acids at positions 237 to 250 of SEQ ID Nos. 33, 35 and 37, the amino acid sequence of the amino acids at positions 227 to 260 of SEQ ID Nos. 33, 35 and 37, the amino acid sequence of the amino acids at positions 217 to 270 of SEQ ID Nos. 33, 35 and 37, and the amino acid sequence of the amino acids at positions 202 to 285 of SEQ ID Nos. 33, 35 and 37. The Zwf protein variants Zwf(R370M) and Zwf(V372A) comprise at least an amino acid sequence selected from the group consisting of the amino acid sequence of the amino acids at positions 365 to 377 of SEQ ID Nos. 29 and 31, the amino acid sequence of the amino acids at positions 355 to 387 of SEQ ID Nos. 29 and 31, the amino acid sequence of the amino acids at positions 345 to 397 of SEQ ID Nos: 29 and 31, and the amino acid sequence of the amino acids at positions 325 to 417 of SEQ ID Nos. 29 and 31. In addition, the Zwf protein variants may comprise a N-terminal amino acid sequence selected from the group consisting of the sequence corresponding to the amino acids at positions 1 to 10 of SEQ ID NO: 10, the amino acid sequence corresponding to the amino acids at positions 1 to 16 of SEQ ID NO: 10, the amino acid sequence corresponding to the amino acids at positions 1 to 20 of SEQ ID NO: 10 and the amino acid sequence corresponding to the amino acids at positions 1 to 30 of SEQ ID NO: 10.

The term proteinogenic amino acid denotes those amino acids which are found in naturally occurring proteins of microorganisms, plants, animals and humans. These amino acids comprise L-glycine, L-alanine, L-valine, L-leucine, L-isoleucine, L-serine, L-threonine, L-cysteine, L-methionine, L-proline, L-phenylalanine, L-tyrosine, L-tryptophan, L-asparagine, L-glutamine, L-aspartic acid, L-glutamic acid, L-arginine, L-lysine, L-histidine and L-selenocysteine.

The replacement of L-alanine in position 243 with L-threonine may preferably be achieved by replacing the nucleobase guanine in position 1264 of SEQ ID NO: 9 with adenine. This guanine/adenine transition is also shown in position 1034 of SEQ ID NO: 21. Positions 1264 of SEQ ID NO: 9 and 1034 of SEQ ID NO: 21 both correspond to position 727 of the coding sequences (the first G of the start codon GTG is position 1 in this case) of the zwf gene and zwf(A243T) allele.

An internal segment of the zwf(A243T) allele is shown in SEQ ID NO: 23. It corresponds to positions 898 to 1653 of SEQ ID NO: 21. The glucose 6-phosphate dehydrogenase activity of the Zwf proteins according to this aspect of the invention is less susceptible or resistant particularly to inhibition by NADPH as compared to the wild type protein. Being exposed to a concentration of approximately 260 μM NADPH, the residual activity is at least 30% or 35%, preferably at least 40%, 45% or 50% as compared to the activity in the absence of added NADPH in a strain comprising the mutant protein. Being exposed to a concentration of approximately 400 μM NADPH the residual activity is at least 20% preferably at least 25% as compared to the activity in the absence of added NADPH.

Mutagenesis to induce mutations or alleles may be performed by conventional mutagenesis methods for bacterial cells using mutagens such as for example N-methyl-N′-nitro-N-nitrosoguanidine or ultraviolet light as described in the art, see e.g., the Manual of Methods for General Bacteriology (Gerhard et al. (Eds.), American Society for Microbiology, Washington, D.C., USA, 1981).

Accordingly, the invention provides isolated coryneform bacteria or mutants comprising a polynucleotide encoding a Zwf protein comprising the amino acid sequence of SEQ ID NO: 10, wherein at least one or more of the amino acids at positions 369 to 373 and/or one or more of the amino acids at positions 241 to 246 is exchanged by another proteinogenic amino acid. Corynebacterium glutamicum DM658 is an example of such a coryneform bacterium. It was obtained after multiple rounds of mutagenesis, selection and screening and contains in its chromosome a zwf allele (zwf(A243T)) coding for a Zwf protein (Zwf(A243T)) having the amino acid sequence of SEQ ID NO: 10 wherein L-alanine at position 243 is replaced by L-threonine as is shown in SEQ ID NO: 22.

Mutagenesis may also be performed using in vitro methods for polynucleotides such as, for example, treatment with hydroxylamine (Molecular and General Genetics 145:101 (1978)), mutagenic oligonucleotides (Brown, Gentechnologie fuer Einsteiger, Spektrum Akademischer Verlag, Heidelberg, 1993), the polymerase chain reaction (PCR), as is described in the manual by Newton et al., (PCR, Spektrum Akademischer Verlag, Heidelberg, 1994), the method described by Papworth, et al. (Strategies 9(3):3-4 (1996)) using the “Quik Change Site-directed Mutagenesis Kit” of Stratagene (La Jolla, Calif., USA), or similar methods known in the art.

The corresponding alleles or mutations are sequenced and introduced by recombination into the chromosome of an appropriate strain by the method of gene replacement, for example as described by Schwarzer, et al. (Bio/Technology 9:84-87 (1991)) for the lysA gene of C. glutamicum or by Peters-Wendisch, et al. (Microbiology 144:915-927 (1998)) for the pyc gene of C. glutamicum. Corynebacterium glutamicum DSM5715zwf2_A243T is an example for such a strain. It comprises in its chromosome the mutation of the zwf allele of strain DM658, i.e. zwf(A243T).

The corresponding alleles can also be introduced into the chromosome of an appropriate strain by the method of gene duplication, for example as described by Reinscheid, et al. (Appl. Environ. Microbiol. 60(1):126-132 (1994)) for the hom-thrB operon or by Jetten, et al. (Appl. Microbiol. Biotech. 43:76-82 (1995)) for the ask gene. Accordingly, the invention further provides coryneform bacteria comprising an isolated polynucleotide encoding a Zwf protein comprising the amino acid sequence of SEQ ID NO: 10, wherein at least one or more of the amino acids at positions 369 to 373 and/or one or more of the amino acids at positions 241 to 246 is exchanged by another proteinogenic amino acid. Corynebacterium glutamicum DSM5715::pK18-mobsacB_zwf(A243T) is an example of such a strain. It comprises in its chromosome an isolated DNA containing the zwf(A243T) allele.

Alleles can also be overexpressed by any of the methods as described above, for example, using plasmids, inducible promoters or any other method known in the art.

The strains thus obtained are used for the fermentative production of amino acids. In addition, it may be advantageous for the production of L-amino acids to amplify one or more enzymes of the particular biosynthesis pathway, of glycolysis, of anaplerosis, of the pentose phosphate pathway or of amino acid export, in addition to amplification of the zwf gene. Thus, for the preparation of L-threonine, one or more genes chosen from the following group may be can be amplified, in particular over-expressed, at the same time:

the hom gene which codes for homoserine dehydrogenase (Peoples, et al, Mol. Microbiol. 2:63-72 (1988)) or the hom^{dr} allele which codes for a “feed back resistant” homoserine dehydrogenase (Archer, et al., Gene 107:53-59 (1991),

the gap gene which codes for glyceraldehyde 3-phosphate dehydrogenase (Eikmanns, et al., J. Bacteriol. 174:6076-6086 (1992)),

the pyc gene which codes for pyruvate carboxylase (Peters-Wendisch, et al., Microbiol. 144:915-927 (1998)),

the mqo gene which codes for malate:quinone oxido-reducctase (Molenaar et al., Eur. J. Biochem. 254:395-403 (1998)),

the tkt gene which codes for transketolase (accession number AB023377 of the European Molecular Biologies Laboratories databank (EMBL, Heidelberg, Germany)),

the gnd gene which codes for 6-phosphogluconate dehydrogenase (JP-A-9-224662),

the thrE gene which codes for the threonine export protein (DE 199 41 478.5; DSM 12840),

the zwal gene (DE 199 59 328.0; DSM 13115),

the eno gene which codes for enolase (DE: 199 41 478.5).

For the preparation of L-lysine, one or more genes chosen from the following group can be amplified, in particular over-expressed, at the same time. The use of endogenous genes is preferred.

the dapA gene which codes for dihydrodipicolinate synthase (EP-B 0 197 335),

the lysC gene which codes for a feed back resistant aspartate kinase (Kalinowski, et al., Mol Gen. Genet. 224:317-324) (1990);

the gap gene which codes for glyceraldehyde 3-phosphate dehydrogenase (Eikmanns J. Bacteriol. 174:6076-6086) (1992),

the pyc gene which codes for pyruvate carboxylase (DE-A-198 31 609),

the tkt gene which codes for transketolase (accession number AB023377 of the European Molecular Biologies Laboratories databank (EMBL, Heidelberg, Germany)),

the gnd gene which codes for 6-phosphogluconate dehydrogenase (JP-A-9-224662),

the lysE gene which codes for the lysine export protein (DE-A-195 48 222),

the zwal gene (DE 199 59 328.0; DSM 13115),

the eno gene which codes for enolase (DE 199 47 791.4)

In addition to the amplification of the zwf gene, the production of L-amino acids can be further enhanced by concurrently attenuating one of the genes chosen from the group consisting of:

the pck gene which codes for phosphoenol pyruvate carboxykinase (DE 199 50 409.1; DSM 13047),

the pgi gene which codes for glucose 6-phosphate isomerase (U.S. Ser. No. 09/396,478, DSM 12969),

the poxB gene which codes for pyruvate oxidase (DE 199 51 975.7; DSM 13114),

the zwa2 gene (DE: 199 59 327.2; DSM 13113)

In this connection, the term “attenuation” means reducing or suppressing the intracellular activity or concentration of one or more enzymes or proteins in a microorganism, which enzymes or proteins are coded by the corresponding DNA. For example, attenuation can be achieved by: using a weak promoter; using a gene or allele which codes for a corresponding enzyme or protein which has a low activity; inactivating the corresponding enzyme or protein; and, optionally, by combining these measures. Using attenuation measures, the activity or concentration of the corresponding enzyme or protein is, in general, reduced to 0 to 75%, 0 to 50%, 0 to 25%, 0 to 10% or 0 to 5% of the activity or concentration of the wild-type enzyme or protein or of the activity or concentration of the enzyme or protein in the starting microorganism.

In addition to amplification of the Zwf protein, it may be advantageous for the production of L-amino acids to eliminate undesirable side reactions (Nakayama: “Breeding of Amino Acid Producing Micro-organisms,” in: Overproduction of Microbial Products, Krumphanzl, Sikyta, Vanek (eds.), Academic Press, London, UK, 1982).

The microorganisms prepared according to the invention can be cultured continuously or discontinuously in a batch process (batch culture), in a fed batch process (feed process), or in a repeated fed batch process (repetitive feed process) for the purpose of L-amino acid production. A summary of known culture methods is described in the textbook by Chmiel (Bioprozesstechnik 1. Einfuehrung in die Bioverfahrenstechnik [Bioprocess Technology 1. Introduction to Bioprocess Technology] (Gustav Fischer Verlag, Stuttgart, 1991)) or in the textbook by Storhas (Bioreaktoren und periphere Einrichtungen [Bioreactors and Peripheral Equipment] Vieweg Verlag, Braunschweig/Wiesbaden, 1994)).

The culture medium must meet the requirements of the particular microorganisms being used. Descriptions of culture media for various microorganisms are contained in the handbook Manual of Methods for General Bacteriology of the American Society for Bacteriology (Washington D.C., USA, 1981). Sugars and carbohydrates (such as glucose, sucrose, lactose, fructose, maltose, molasses, starch and cellulose), oils and fats (such as soy oil, sunflower oil, groundnut oil and coconut fat), fatty acids (such as palmitic acid, stearic acid and linoleic acid), alcohols (such as glycerol and ethanol) and organic acids (such as acetic acid) can be used as the source of carbon. These substance can be used individually or as a mixture.

Organic nitrogen-containing compounds, such as peptones, yeast extract; meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can also be used individually or as a mixture.

Potassium dihydrogen phosphate or dipotassium hydrogen phosphate or the corresponding sodium-containing salts can be used as the source of phosphorus.

The culture medium must furthermore comprise salts of metals, such as magnesium sulfate or iron sulfate, which are necessary for growth. Finally, essential growth substances, such as amino acids and vitamins, can be employed in addition to the above-mentioned substances.

Suitable precursors can be added to the culture medium. The starting substances mentioned can be added to the culture in the form of a single batch, or can be fed in during the culture in a suitable manner. Basic compounds, such-as sodium hydroxide, potassium hydroxide, ammonia, or acid compounds, such as phosphoric acid or sulfuric acid, can be employed in a suitable manner to control the pH. Antifoams, such as fatty acid polyglycol esters, can be employed to control the development of foam. Suitable substances having a selective action, e.g., antibiotics, can also be added to the medium to maintain the stability of plasmids.

To maintain aerobic conditions, oxygen or oxygen-containing gas mixtures, such as air, are introduced into the culture. The temperature of the culture is usually 20° C. to 45° C., and preferably 25° C. to 40° C. Culturing is continued until a maximum of L-amino acid has formed. This target is usually reached within 10 hours to 160 hours.

Accordingly, the invention provides a process for the preparation of an amino acid by fermentation of a coryneform bacterium, comprising the following steps:

a) fermenting an amino acid producing bacterium in which at least a zwf gene encoding the Zwischenferment protein is overexpressed, and

b) concentrating the amino acid in the medium or in the cells of the bacteria wherein said Zwischenferment protein comprises at least the amino acid sequence corresponding to amino acids at positions 241 to 246 of SEQ ID NO: 22 and optionally the N terminal amino acid sequence of SEQ ID NO: 10 amino acids 1 to 10 or SEQ ID NO: 10 amino acids 2 to 10.

The invention further provides a process for the preparation of an amino acid by fermentation of an isolated coryneform bacterium comprising the following steps:

a) fermenting an amino acid producing bacterium comprising a polynucleotide encoding a Zwf protein with the amino acid sequence of SEQ ID NO: 10, wherein at least one or more of the amino acids at positions 369 to 373 and/or one or more of the amino acids at positions 241 to 246 is exchanged by another proteinogenic amino acid, and

b) concentrating of the amino acid in the medium or in the cells of the bacterium.

The invention also provides a process for the preparation of an amino acid by fermentation of a coryneform bacterium comprising the following steps:

a) fermenting an amino acid producing bacterium comprising an isolated polynucleotide encoding a Zwf protein comprising the amino acid sequence of SEQ ID NO: 10, wherein at least one or more of the amino acids at positions 369 to 373 and/or one or more of the amino acids at positions 241 to 246 is exchanged by another proteinogenic amino acid, and

b) concentrating of the amino acid in the medium or in the cells of the bacterium.

The amino acids produced by the methods described above may be isolated from the medium or the bacterial cells. Analysis of L-amino acids can be carried out by anion exchange chromatography with subsequent ninhydrin derivation, as described by Spackman et al. (Anal. Chem. 30:1190 (1958)), or by reversed phase HPLC as described by Lindroth et al. (Anal. Chem. 51:1167-1174 (1979)).

The following microorganisms have been deposited as pure cultures at the Deutsche Sammlung fuer Mikroorganismen und Zellkulturen (DSMZ=German Collection of Microorganisms and Cell Cultures, Braunschweig, Germany):

Escherichia coli K-12 DH5α/pEC-T18mob2 was deposited on Jan. 20, 2000 as DSM 13244 in accordance with the Budapest Treaty.

Corynebacterium glutamicum DM658 was deposited on Jan. 27, 1993 as DSM 7431 for long term storage. This deposition was converted to a deposition in accordance with the Budapest Treaty on Oct. 17, 2002.

Corynebacterium glutamicum DSM5715zwf2_A243T was deposited on Oct. 11, 2002 as DSM 15237 in accordance with the Budapest Treaty.

The following examples will further illustrate the present invention. The molecular biology techniques, e.g., plasmid DNA isolation, restriction enzyme treatment, ligations, standard transformations of Escherichia coli etc. used are, (unless stated otherwise), described by Sambrook et al., (Molecular Cloning. A Laboratory Manual (1989) Cold Spring Harbor Laboratories, USA).

### EXAMPLES

### Example 1

Expression of the Zwf Protein

1.1 Preparation of the Plasmid pEC-T18mob2

The E. coli—C. glutamicum shuttle vector pEC-T18mob2 was constructed according to the prior art. The vector contains the replication region, rep, of the plasmid pGA1 including the replication effector per (U.S. Pat. No. 5,175,108; Nesvera et al., J. Bacteriol. 179:1525-1532 (1997)), the tetracycline resistance-imparting tetA(Z) gene of the plasmid pAG1 (U.S. Pat. No. 5,158,891; gene library entry at the National Center for Biotechnology Information, NCBI, Bethesda, Md., USA, accession number AF121000), the replication region oriV of the plasmid pMB1 (Sutcliffe, Cold Spring Harbor Symp. Quant. Biol. 43:77-90 (1979)), the lacZα gene fragment including the lac promoter and a multiple cloning site (mcs) (Norrander, et al., Gene 26:101-106 (1983)) and the mob region of the plasmid RP4 (Simon, et al, Bio/Technology 1:784-791 (1983)). The vector constructed was transformed in the E. coli strain DH5α (Brown (ed.) Molecular Biology Labfax, BIOS Scientific Publishers, Oxford, UK, 1991). Selection for plasmid-carrying cells was made by plating out the transformation batch on LB agar (Sambrook, et al., Molecular Cloning: A Laboratory Manual, 2^{nd} ed., Cold Spring Harbor Laboratory Press, Cold Spring Harbor, N.Y.), which had been supplemented with 5 mg/l tetracycline. Plasmid DNA was isolated from a transformant with the aid of the QIAprep Spin Miniprep Kit from Qiagen and checked by restriction with the restriction enzyme EcoRI and HindIII and subsequent agarose gel electrophoresis (0.8%).

The plasmid was called pEC-T18mob2 and is shown in FIG. 1. It is deposited in the form of the strain Escherichia coli K-12 strain DH5αpEC-T18mob2 at the Deutsche Sammlung fuer Mikroorganismen und Zellkulturen (DSMZ=German Collection of Microorganisms and Cell Cultures, Braunschweig, Germany) as DSM 13244.

1.2 Preparation of the Plasmid pEC-T18mob2zwf

The zwf gene from Corynebacterium glutamicum ATCC13032 was first amplified by a polymerase chain reaction (PCR) by means of the following oligonucleotide primers:

| | |
|---|---|
| zwf-forward: | |
| 5′-TCG ACG CGG TTC TGG AGC AG-3′ | (SEQ ID NO 11) |
| zwf-reverse: | |
| 5′-CTA AAT TAT GGC CTG CGC CAG-3′ | (SEQ ID NO 12) |

The PCR reaction was carried out in 30 cycles in the presence of 200 μM deoxynucleotide triphosphates (dATP, dCTP, dGTP, dTTP), in each case 1 μM of the corresponding oligonucleotide, 100 nanogram (ng) chromosomal DNA from Corynebacterium glutamicum ATCC13032, 1/10 volume 10-fold reaction buffer and 2.6 units of a heat-stable Taq-/Pwo-DNA polymerase mixture (Expand High Fidelity PCR System from Roche Diagnostics, Mannheim, Germany) in a Thermocycler (PTC-100, MJ Research, Inc., Watertown, USA) under the following conditions: 94° C. for 30 seconds, 64° C. for 1 minute and 68° C. for 3 minutes.

The amplified fragment about 1.8 kb in size was subsequently ligated with the aid of the SureClone Ligation Kit (Amersham Pharmacia Biotech, Uppsala, Sweden) into the SmaI cleavage site of the vector pUC18 in accordance with the manufacturer's instructions. The E. coli strain DH5ocmcr (Grant, et al., Proc. Nat'l Acad. Sci. USA 87:4645-4649 (1990)) was transformed with the entire ligation batch. Transformants were identified based upon their carbenicillin resistance on LB-agar plates containing 50 μg/mL carbenicillin. The plasmids were prepared from 7 of the transformants and checked for the presence of the 1.8 kb PCR fragment as an insert by restriction analysis. The recombinant plasmid formed in this way is called pUC18zwf.

For construction of pEC-T18mob2zwf, pUC18zwf was digested with KpnI and SalI, and the product was isolated with the aid of the NucleoSpin Extraction Kit from Macherey-Nagel (Dueren, Germany) in accordance with the manufacturer's instructions and then ligated with the vector pEC-T18mob2, which had also been cleaved with KpnI and SalI and dephosphorylated. The E. coli strain DH5αmcr was transformed with the entire ligation batch. Transformants were identified based upon their tetracycline resistance on LB-agar plates containing 5 μg/mL tetracycline. The plasmids were prepared from 12 of the transformants and checked for the presence of the 1.8 kb PCR fragment as an insert by restriction analysis. One of the recombinant plasmids isolated in this manner was called pEC-T18mob2zwf (FIG. 2).

### Example 2

Preparation of Amino Acid Producers with an Amplified zwf Gene

The L-lysine-producing strain Corynebacterium glutamicum DSM5715 is described in EP-B-0435132 and the L-threonine-producing strain Brevibacterium flavum DSM5399 is described in EP-B-0385940. Both strains are deposited at the Deutsche Sammlung fuer Mikroorganismen und Zellkulturen [German Collection of Microorganisms and Cell Cultures] in Braunschweig (Germany) in accordance with the Budapest Treaty.

2.1 Preparation of the Strains DSM5715/pEC-T18mob2zwf and DSM5399/pEC-T18mob2zwf

The strains DSM5715 and DSM5399 were transformed with the plasmid pEC-T18mob2zwf using the electroporation method described by Liebl et al., (FEMS Microbiol. Lett. 53:299-303 (1989)). Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 5 mg/l tetracycline. Incubation was carried out for 2 days at 33° C. Plasmid DNA was isolated in each case from a transformant by conventional methods (Peters-Wendisch, et al., Microbiol. 144:915-927 (1998)), cleaved with the restriction endonucleases XbaI and KpnI, and the plasmid was checked by subsequent agarose gel electrophoresis. The strains obtained in this way were called DSM5715/pEC-T18mob2zwf and DSM5399/pEC-T18mob2zwf.

2.2 Preparation of L-Threonine

The C. glutamicum strain DSM5399/pEC-T18mob2zwf obtained in Example 2.1 was cultured in a nutrient medium suitable for the production of threonine and the threonine content in the culture supernatant was determined. For this, the strain was first incubated on an agar plate with the corresponding antibiotic (brain-heart agar with tetracycline (5 mg/l)) for 24 hours at 33° C. Starting from this agar plate culture, a preculture was seeded (10 ml medium in a 100 ml conical flask). The complete medium Cg III was used as the medium for the preculture.

Medium Cg III

| | | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |
| The pH was brought to pH 7.4 | | |

Tetracycline (5 mg/l) was added to this. The preculture was incubated for 16 hours at 33° C. at 240 rpm on a shaking machine. A main culture was seeded from this preculture such that the initial OD (660nm) of the main culture was 0.1. Medium MM was used for the main culture.

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 50 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) was added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of threonine formed was determined with an amino acid analyzer from Eppendorf-BioTronik (Hamburg, Germany) by ion exchange chromatography and post-column derivation with ninhydrin detection. The result of the experiment is shown in Table 1.

TABLE 1

| | OD | L-Threonin |
|---|---|---|
| Strain | (660 nm) | g/l |
| DSM5399 | 12.3 | 0.74 |
| DSM5399/pEC-T18mob2zwf | 10.2 | 1.0 |

2.3 Preparation of L-Lysine

The C. glutamicum strain DSM5715/pEC-T18mob2zwf obtained in Example 2.1 was cultured in a nutrient medium suitable for the production of lysine and the lysine content in the culture supernatant was determined. For this, the strain was first incubated on an agar plate with the corresponding antibiotic (brain-heart agar with tetracycline (5 mg/l)) for 24 hours at 33° C. Starting from this agar plate culture, a preculture was seeded (10 ml medium in a 100 ml conical flask). The complete medium Cg III was used as the medium for the preculture.

Medium Cg III

| | | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |
| The pH was brought to pH 7.4 | | |

Tetracycline (5 mg/l) was added to this. The preculture was incubated for 16 hours at 33° C. at 240 rpm on a shaking machine. A main culture was seeded from this preculture such that the initial OD (660nm) of the main culture was 0.1. Medium MM was used for the main culture.,

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 58 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) was added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivation with ninhydrin detection. The result of the experiment is shown in Table 2.

TABLE 2

| | OD | L-Lysine HCl |
|---|---|---|
| Strain | (660 nm) | g/l |
| DSM5715 | 10.8 | 16.0 |
| DSM5715/pEC-T18mob2zwf | 7.2 | 17.1 |

### Example 3

Construction of a Gene Library of Corynebacterium glutamicum Strain AS019

A DNA library of Corynebacterium glutamicum strain AS019 (Yoshihama, et al., J. Bacteriol. 162:591-597 (1985)) was constructed using λ Zap Express™ system, (Short, et al., Nucl. Ac. Res. 16:7583-7600 (1988)), as described by O'Donohue (O'Donohue, M., The Cloning and Molecular Analysis of Four Common Aromatic Amino Acid Biosynthetic Genes from Corynebacterium glutamicum, Ph.D. Thesis, National University of Ireland, Galway (1997)). λ Zap Express™ kit was purchased from Stratagene (Stratagene, 11011 North Torrey Pines Rd., La Jolla, Calif. 92037) and used according to the manufacturers instructions. AS019-DNA was digested with restriction enzyme Sau3A and ligated to BamHI treated and dephosphorylated λ Zap Express arms.

### Example 4

Cloning and Sequencing of the pgi Gene

4.1 Cloning

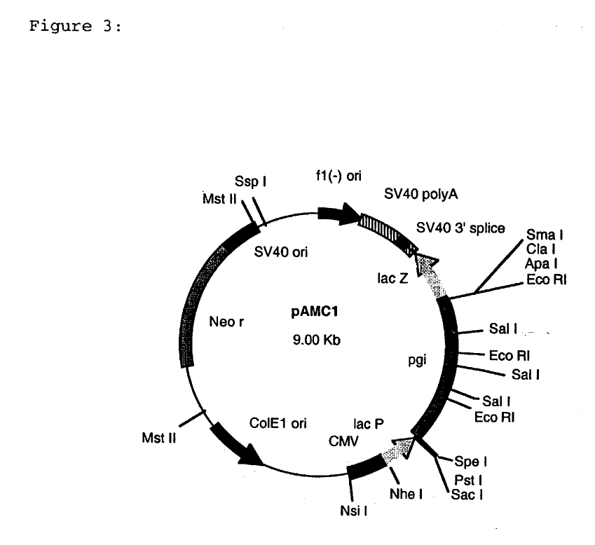

Escherichia coli strain DF 1311, carrying mutations in the pgi and pgl genes as described by Kupor, et al. (J. Bacteriol. 100:1296-1301 (1969)), was transformed with approx. 500 ng of the AS019 λ Zap Express™ plasmid library described in Example 3. Selection for transformants was made on M9 minimal media, (Sambrook et al., Molecular Cloning. A Laboratory Manual, Cold Spring Harbor Laboratories, USA (1989)), containing kanamycin at a concentration of 50 mg/l and incubation at 37° C. for 48 hours. Plasmid DNA was isolated from one transformant according to Bimboim et al. (Nucl. Ac. Res. 7:1513-1523 (1979)) and designated pAMC1 (FIG. 3).

4.2 Sequencing

For sequence analysis of the cloned insert of pAMC1 the method of Sanger, et al. (Proc. Nat'l Acad. Sci. USA 74:5463-5467 (1977)) was applied using primers differentially labeled with a colored fluorescent tag. It was carried out using the ABI prism 310 genetic analyzer from Perkin Elmer Applied Biosystems, (Perkin Elmer Corporation, Norwalk, Connecticut, U.S.A), and the ABI prism Big Dye™ Terminator Cycle Sequencing Ready Reaction kit also from Perkin Elmer.

Initial sequence analysis was carried out using the universal forward and M13 reverse primers obtained from Pharmacia Biotech (St. Albans, Herts, AL1 3AW, UK): Universal forward primer: GTA ATA CGA CTC ACT ATA GGG C (SEQ ID NO: 13) M13 reverse primer: GGA AAC AGC TAT GAC CAT G (SEQ ID NO 14)

Internal primers were subsequently designed from the sequence obtained which allowed the entire pgi gene to be deduced. The sequence of the internal primers is as follows:

| | |
|---|---|
| Internal primer 1: | |
| GGA AAC AGG GGA GCC GTC | (SEQ ID NO 15) |
| Internal primer 2: | |
| TGC TGA GAT ACC AGC GGT | (SEQ ID NO 16) |

The sequence obtained was then analyzed using the DNA Strider program, (Marck, Nucl. Ac. Res. 16:1829-1836 (1988)), version 1.0 on an Apple Macintosh computer. This program allowed for analyses such as restriction site usage, open reading frame analysis and codon usage determination. Searches between DNA sequences obtained and those in EMBL and GenBank databases were achieved using the BLAST program, (Altschul et al., Nucl. Ac. Res. 25:3389-3402 (1997)). DNA and protein sequences were aligned using the Clustal V and Clustal W programs (Higgins et al., Gene 73:237-244 (1988)). The sequence thus obtained is shown in SEQ ID NO: 1. The analysis of the nucleotide sequence obtained revealed an open reading frame of 1650 base pairs which was designated as the pgi gene. It codes for a protein of 550 amino acids shown in SEQ ID NO: 2.

### Example 5

Preparation of an Integration Vector for Integration Mutagenesis of the pgi Gene

An internal segment of the pgi gene was amplified by polymerase chain reaction (PCR) using genomic DNA isolated from Corynebacterium glutamicum AS019, (Heery at al, Appl. Envir. Microbiol. 59:791-799 (1993)), as template. The pgi primers used were:

| | |
|---|---|
| fwd. Primer: | |
| ATG GAR WCC AAY GGH AA | (SEQ ID NO 17) |
| rev. Primer: | |
| YTC CAC GCC CCA YTG RTC | (SEQ ID NO 18) |

with R = A + G; Y = C + T; W = A + T; H = A +
T + C.

PCR Parameters were as follows: 35 cycles

94° C. for 1 min.

47° C. for 1 min.

72° C. for 30 sec.

1.5 mM MgCl₂

approx. 150-200 ng DNA template.

The PCR product obtained was cloned into the commercially available pGEM-T vector received from Promega Corp., (Promega UK, Southampton.) using strain E. coli JM109 (Yanisch-Perron, et al., Gene 33:103-119 (1985)) as a host. The sequence of the PCR product is shown as SEQ ID NO: 3. The cloned insert was then excised as an EcoRI fragment and ligated to plasmid pBGS8 (Spratt, et al., Gene 41:337-342 (1986)) pretreated with EcoRI. The restriction enzymes used were obtained from Boehringer Mannheim UK Ltd., (Bell Lane, Lewes East Sussex BN7 1 LG, UK.) and used according to manufacturers instructions. E. coli JM109 was then transformed with this ligation mixture and electrotransformants were selected on Luria agar supplemented with IPTG (isopropyl-β-D-thiogalactopyranoside), XGAL (5-bromo-4-chloro-3-indolyl-D-galactopyranoside) and kanamycin at a concentration of 1 mM, 0.02% and 50 mg/l, respectively.

Agar plates were incubated for twelve hours at 37° C. Plasmid DNA was isolated from one transformant, characterized by restriction enzyme analysis using EcoRI, BamHI and SalI designated pMC1 (FIG. 4). Plasmid pMC1 was deposited in the form of Escherichia coli strain DHSa/pMC1 at the Deutsche Sammlung fuer Mikroorganismen und Zellkulturen (DSMZ, Braunschweig, Germany) as DSM 12969 according to the Budapest treaty.

### Example 6

Integration Mutagenesis of the pgi Gene in the Lysine Producer DSM 5715

The vector pMC1 mentioned in Example 5 was electroporated by the electroporation method of Tauch et al. (FEMS Microbiol. Lett. 123:343-347 (1994)) in Corynebacterium glutamicum DSM 5715. Strain DSM 5715 is an AEC-resistant lysine producer. The vector pMC1 cannot replicate independently in DSM5715 and is retained in the cell only if it has integrated into the chromosome of DSM 5715. Selection of clones with pMC1 integrated into the chromosome was carried out by plating out the electroporation batch on LB agar (Sambrook, et al., Molecular Cloning: A Laboratory Manual, 2^{nd} ed., Cold Spring Harbor Laboratory Press, Cold Spring Harbor, N.Y.), which had been supplemented with 15 mg/l kanamycin.

For detection of the integration, the internal pgi fragment (Example 5) was labeled with the Dig hybridization kit from Boehringer Mannheim by the method of “The DIG System Users Guide for Filter Hybridization” of Boehringer Mannheim GmbH (Mannheim, Germany, 1993). Chromosomal DNA of a transformant was isolated by the method of Eikmanns et al. (Microbiol. 140:1817-1828 (1994)) and in each case cleaved with the restriction enzymes SalI, SadI and HindIII. The fragments formed were separated by agarose gel electropboresis and hybridized at 68° C. with the Dig hybridization kit from Boehringer. It was found in this way that the plasmid pMC1 was inserted within the chromosomal pgi gene of strain DSM5715. The strain was called DSM5715::pMC1.

### Example 7

Effect of Over-Expression of the zwf Gene with Simultaneous Elimination of the pgi Gene on the Preparation of Lysine

7.1 Preparation of the Strain DSM5715::pMC1/pEC-T18mob2zwf

The vector pEC-T18mob2zwf mentioned in Example 1.2 was electroporated by the method of Tauch et al. (FEMS Microbiol. Lett. 123:343-347 (1994)) in Corynebacterium glutamicum DSM 5715::pMC1. Selection for plasmid-carrying cells was made by plating out the electroporation batch on LB agar (Sambrook, et al., Molecular Cloning: A Laboratory Manual, 2^{nd} ed., Cold Spring Harbor Laboratory Press, Cold Spring Harbor, N.Y., 1989), which had been supplemented with 15 mg/l kanamycin and with 5 mg/l tetracycline. Plasmid DNA was isolated from a transformant by conventional methods (Peters-Wendisch et al., Microbiol. 144:915-927 (1998)) and checked by treatment with the restriction enzymes KpnI and SalI with subsequent agarose gel electrophoresis. The strain was called DSM5715::pMC1/pEC-T18mob2zwf.

7.2 Preparation of Lysine

The C. glutamicum strain DSM5715::pMC1/pEC-T18mob2zwf obtained in Example 7.1 was cultured in a nutrient medium suitable for the production of lysine and the lysine content in the culture supernatant was determined. For this, the strain was first incubated on an agar plate with the corresponding antibiotic (brain-heart agar with tetracycline (5 mg/l) and kanamycin (25 mg/l)) for 24 hours at 33° C. The cultures of the comparison strains were supplemented according to their resistance to antibiotics. Starting from this agar plate culture, a preculture was seeded (10 ml medium in a 100 ml conical flask). The complete medium Cg III was used as the medium for the preculture.

Medium Cg III

| | | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |
| The pH was brought to pH 7.4 | | |

Tetracycline (5 mg/l) and kanamycin (5 mg/l) was added to this. The preculture was incubated for 16 hours at 33° C. at 240 rpm on a shaking machine. A main culture was seeded from this preculture such that the initial OD (660 nm) of the main culture was 0.1. Medium MM was used for the main culture.

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 50 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) and kanamycin (25 mg/l) were added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivation with ninhydrin detection. The result of the experiment is shown in Table 3.

TABLE 3

| | OD | L-Lysine HCl |
|---|---|---|
| Strain | (660 nm) | g/l |
| DSM5715 | 7.3 | 14.3 |
| DSM5715/pEC-T18mob2zwf | 7.1 | 14.6 |
| DSM5715::pMC1/ | 10.4 | 15.2 |
| pECTmob2zwf | | |

### Example 8

Preparation of a Genomic Cosmid Gene Library from Corynebacterium glutamicum ATCC 13032

Chromosomal DNA from Corynebacterium glutamicum ATCC 13032 was isolated as described by Tauch et al., (Plasmid 33:168-179 (1995)), and partly cleaved with the restriction enzyme Sau3AI (Amersham Pharmacia, Freiburg, Germany, Product Description Sau3AI, Code no. 27-0913-02). The DNA fragments were dephosphorylated with shrimp alkaline phosphatase (Roche Molecular Biochemicals, Mannheim, Germany, Product Description SAP, Code no. 1758250). The DNA of the cosmid vector SuperCosl (Wahl, et al., Proc. Nat'l Acad. Sci. USA 84:2160-2164 (1987)), obtained from Stratagene (La Jolla, USA, Product Description SuperCos1 Cosmid Vektor Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase.

The cosmid DNA was then cleaved with the restriction enzyme BamHI (Amersham Pharmacia, Freiburg, Germany, Product Description BamHI, Code no. 27-0868-04). Cosmid DNA treated in this manner was mixed with the treated ATCC13032 DNA and the batch was treated with T4 DNA ligase (Amersham Pharmacia, Freiburg, Germany, Product Description T4-DNA-Ligase, Code no.27-0870-04). The ligation mixture was then packed in phages with the aid of Gigapack II XL Packing Extracts (Stratagene, La Jolla, USA, Product Description Gigapack II XL Packing Extract, Code no. 200217). For infection of the E. coli strain NM554 (Raleigh, et al., Nucl. Ac. Res. 16:1563-1575 (1988)), the cells were taken up in 10 mM MgSO₄ and mixed with an aliquot of the phage suspension. The infection and titering of the cosmid library were carried out as described by Sambrook et al. (Molecular Cloning: A laboratory Manual, Cold Spring Harbor (1989)), the cells being plated out on LB agar (Lennox, Virology 1:190 (1955))+100 μg/ml ampicillin. After incubation overnight at 37° C., recombinant individual clones were selected.

### Example 9

Isolation and Sequencing of the poxB Gene

The cosmid DNA of an individual colony (Example 7) was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Molecular Biochemicals, Mannheim; Germany, Product Description SAP, Product No. 1758250). After separation by gel electrophoresis, cosmid fragments in the size range of 1500 to 2000 bp were isolated with the QiaExII Gel Extraction Kit (Product No. 20021, Qiagen, Hilden, Germany).

The DNA of the sequencing vector pZero- 1, obtained from Invitrogen (Groningen, Holland, Product Description Zero Background Cloning Kit, Product No. K2500-01), was cleaved with the restriction enzyme BamHI (Amersham Pharmacia, Freiburg, Germany, Product Description BamHI, Product No. 27-0868-04). The ligation of the cosmid fragments in the sequencing vector pZero-1 was carried out as described by Sambrook et al. (Molecular Cloning: A laboratory Manual, Cold Spring Harbor 1989), the DNA mixture being incubated overnight with T4 ligase (Pharmacia Biotech, Freiburg, Germany).

This ligation mixture was then electroporated (Tauch, et al., FEMS Microbiol Lett. 123:343-7 (1994)) into the E. coli strain DH5αMCR (Grant, Proc. Nat'l Acad. Sci. USA 87:4645-4649 (1990)) and plated out on LB agar (Lennox, Virology 1:190 (1955)) with 50 μg/ml zeocin. The plasmid preparation of the recombinant clones was carried out with Biorobot 9600 (Product No. 900200, Qiagen, Hilden, Germany). The sequencing was carried out by the dideoxy chain-stopping method of Sanger et al. (Proc. Nat'l Acad. Sci. USA 74:5463-5467 (1977)) with modifications according to Zimmermann et al. (Nucl. Ac. Res. 18:1067 (1990)). The “RR dRhodamin Terminator Cycle Sequencing Kit” from PE Applied Biosystems(Product No. 403044, Weiterstadt, Germany) was used. The separation by gel electrophoresis and analysis of the sequencing reaction were carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained was processed using the Staden program package (Nucl. Ac. Res. 14:217-231 (1986)) version 97-0. The individual sequences of the pZero1 derivatives were assembled to a continuous contig. The computer-assisted coding region analysis was prepared with the XNIP program (Staden, Nucl. Ac. Res. 14:217-231 (1986)). Further analyses were carried out with the “BLAST search program” (Altschul, et al., Nucl. Ac. Res. 25:3389-3402 (1997)), against the non-redundant databank of the “National Center for Biotechnology Information” (NCBI, Bethesda, Md., USA).

The resulting nucleotide sequence is shown in SEQ ID NO: 4. Analysis of the nucleotide sequence showed an open reading frame of 1737 base pairs, which was called the poxB gene. The poxB gene codes for a polypeptide of 579 amino acids (SEQ ID NO: 5).

### Example 10

Preparation of an Integration Vector for Integration Mutagenesis of the poxB Gene

From the strain ATCC 13032, chromosomal DNA was isolated by the method of Eikmanns et al. (Microbiol. 140:1817-1828 (1994)). On the basis of the sequence of the poxB gene known for C. glutamicum from Example 8, the following oligonucleotides were chosen for the polymerase chain reaction:

| | |
|---|---|
| poxBint1: | |
| 5′ TGC GAG ATG GTG AAT GGT GG 3′ | (SEQ ID NO 19) |
| poxBint2: | |
| 5′ GCA TGA GGC AAC GCA TTA GC 3′ | (SEQ ID NO 20) |

The primers shown were synthesized by MWG Biotech (Ebersberg, Germany) and the PCR reaction was carried out by the standard PCR method of Innis, et al. (PCR protocols. A guide to methods and applications, Academic Press (1990)) with Pwo-Polymerase from Boehringer. With the aid of the polymerase chain reaction, a DNA fragment approx. 0.9 kb in size was isolated, this carrying an internal fragment of the poxB gene and is shown as SEQ ID NO: 6.

The amplified DNA fragment was ligated with the TOPO TA Cloning Kit from Invitrogen Corporation (Carlsbad, Calif., USA; Catalogue Number K4500-01) in the vector pCR2.1-TOPO (Mead, et al., Bio/Technology 9:657-663 (1991)). The E. coli strain DH5α was then electroporated with the ligation batch (Hanahan, In: DNA cloning. A Practical Approach. Vol. I, IRL-Press, Oxford, Washington D.C., USA, 1985). Selection for plasmid-carrying cells was made by plating out the transformation batch on LB agar (Sambrook, et al., Molecular Cloning: A Laboratory Manual, 2^{nd} ed., Cold Spring Harbor Laboratory Press, Cold Spring Harbor, N.Y., 1989), which had been supplemented with 25 mg/l kanamycin. Plasmid DNA was isolated from a transformant with the aid of the QIAprep Spin Miniprep Kit from Qiagen and checked by restriction with the restriction enzyme EcoRI and subsequent agarose gel electrophoresis (0.8%). The plasmid was called pCR2.1poxBint (FIG. 5).

Plasmid pCR2.1poxBint has been deposited in the form of the strain Escherichia coli DH5α/pCR2.1poxBint as DSM 13114 at the Deutsche Sammlung fuer Mikroorganismen und Zellkulturen (DSMZ=German Collection of Microorganisms and Cell Cultures, Braunschweig, Germany) in accordance with the Budapest Treaty.

### Example 11

Integration Mutagenesis of the poxB Gene in the Lysine Producer DSM 5715

The vector pCR2.1poxBint mentioned in Example 10 was electroporated by the electroporation method of Tauch et al. (FEMS Microbiol. Lett. 123:343-347 (1994)) in Corynebacterium glutamicum DSM 5715. Strain DSM 5715 is an AEC-resistant lysine producer. The vector pCR2.1poxBint cannot replicate independently in DSM5715 and is retained in the cell only if it has integrated into the chromosome of DSM 5715. Selection of clones with pCR2.1poxBint integrated into the chromosome was carried out by plating out the electroporation batch on LB agar (Sambrook, et al., Molecular Cloning: A Laboratory Manual, 2^{nd} ed., Cold Spring Harbor Laboratory Press, Cold Spring Harbor, N.Y.), which had been supplemented with 15 mg/l kanamycin. For detection of the integration, the poxBint fragment was labeled with the Dig hybridization kit from Boehringer by the method of “The DIG System Users Guide for Filter Hybridization” of Boehringer Mannheim GmbH (Mannheim, Germany, 1993).

Chromosomal DNA of a potential integrant was isolated by the method of Eikmanns et al. (Microbiol. 140:1817-1828 (1994)) and in each case cleaved with the restriction enzymes SalI, SacI and HindIII. The fragments formed were separated by agarose gel electrophoresis and hybridized at 68° C. with the Dig hybridization kit from Boehringer. The plasmid pCR2.1poxBint mentioned in Example 9 had been inserted into the chromosome of DSM5715 within the chromosomal poxB gene. The strain was called DSM5715::pCR2.1 poxBint.

### Example 12

Effect of Over-Expression of the zwf Gene with Simultaneous Elimination of the poxB Gene on the Preparation of Lysine

12.1 Preparation of the Strain DSM5715::pCR2.1poxBint/pEC-T18mob2zwf The strain DSM5715::pCR2.1poxBint was transformed with the plasmid pEC-T18mob2zwf using the electroporation method described by Liebl et al., (FEMS Microbiol. Lett. 53:299-303 (1989)). Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 5 mg/l tetracycline and 25 mg/l kanamycin. Incubation was carried out for 2 days at 33° C.

Plasmid DNA was isolated in each case from a transformant by conventional methods (Peters-Wendisch, et al., 1998, Microbiol. 144:915-927 (1998)), cleaved with the restriction endonucleases XbaI and KpnI, and the plasmid was checked by subsequent agarose gel electrophoresis. The strain obtained in this way was called DSM5715:pCR2.1poxBint/pEC-T18mob2zwf.

12.2 Preparation of L-Lysine

The C. glutamicum strain DSM5715::pCR2.1poxBint/pEC-T18mob2zwf obtained in Example 12.1 was cultured in a nutrient medium suitable for the production of lysine and the lysine content in the culture supernatant was determined. For this, the strain was first incubated on an agar plate with the corresponding antibiotic (brain-heart agar with tetracycline (5 mg/l) and kanamycin (25 mg/l)) for 24 hours at 33° C. The comparison strains were supplemented according to their resistance to antibiotics. Starting from this agar plate culture, a preculture was seeded (10 ml medium in a 100 ml conical flask). The complete medium Cg III was used as the medium for the preculture.

Medium Cg III

| | | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |
| The pH was brought to pH 7.4 | | |

Tetracycline (5 mg/l) and kanamycin (25 mg/l) were added to this. The preculture was incubated for 16 hours at 33° C. at 240 rpm on a shaking machine. A main culture was seeded from this preculture such that the initial OD (660 nm) of the main culture was 0.1. Medium MM was used for the main culture.

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 58 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) and kanamycin (25 mg/l) were added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivation with ninhydrin detection. The result of the experiment is shown in Table 4.

TABLE 4

| | OD | L-Lysine HCl |
|---|---|---|
| Strain | (660 nm) | g/l |
| DSM5715 | 10.8 | 16.0 |
| DSM5715/pEC-T18mob2zwf | 8.3 | 17.1 |
| DSM5715::pCR2.1poxBint | 7.1 | 16.7 |
| DSM5715::pCR2.1poxBint/ | 7.8 | 17.7 |
| pEC-Tmob2zwf | | |

### Example 13

The zwf allele zwf(A243T)

Isolation and Sequencing

The Corynebacterium glutamicum strain DM658 was prepared by multiple, non-directed mutagenesis, mutant selection and screening from C. glutamicum ATCC13032. The strain is resistant against the L-lysine analogue S-(2-aminoethyl)-L-cysteine (AEC) and has a feedback resistant aspartate kinase which is insensitive to mixtures of L-lysine, the L-lysine analogue S-(2-aminoethyl)-L-cysteine (AEC) and L-threonine. Strain DM658 is deposited at the Deutsche Sammlung fuer Mikroorganismen und Zellkulturen (DSMZ=German Collection of Microorganisms and Cell under DSM7431.

From the strain DM658, chromosomal DNA is isolated by conventional methods (Eikmanns et al., Microbiol. 140:1817-1828 (1994)). With the aid of the polymerase chain reaction (PCR), a DNA section which carries the zwf gene or allele is amplified. On the basis of the sequence of the zwf gene of C. glutamicum the following primer oligonucleotides from Example 1.2 are chosen for the PCR:

| | |
|---|---|
| zwf-forward: | |
| 5′-TCG ACG CGG TTC TGG AGC AG-3′ | (SEQ ID NO 11) |
| zwf-reverse: | |
| 5′-CTA AAT TAT GGC CTG CGC CAG-3′ | (SEQ ID NO 12) |

The primers shown are synthesized by MWG Biotech (Ebersberg, Germany) and the PCR reaction is carried out by the standard PCR method of Innis, et al. (PCR Protocols. A Guide to Methods and Applications, Academic Press (1990)). The primers allow amplification of a DNA section of approximately 1.85 kb in length, which carries the zwf allele. The amplified DNA fragment of approx. 1.85 kb in length which carries the zwf allele of strain DM658 is identified by electrophoresis in a 0.8% agarose gel, isolated from the gel and purified by conventional methods (QIAquick Gel Extraction Kit, Qiagen, Hilden, Germany). The nucleotide sequence of the amplified DNA fragment or PCR product is determined by sequencing by MWG Biotech (Ebersberg, Germany). The sequence of the PCR product is shown in SEQ ID NO: 21. The amino acid sequence of the Zwischenferment protein (Zwf protein) resulting with the aid of the Patentin program is shown in SEQ ID NO: 22.

The nucleotide sequence of the coding region of the zwf allele of strain DM658 contains at position 727 the base adenine. The position 727 of the nucleotide sequence in the coding region of the zwf-allele corresponds to position 1034 of the nucleotide sequence shown in SEQ ID NO: 21. At position 727 of the nucleotide sequence of the coding region of the wild-type gene the nucleotide is the base guanine. The position 727 of the nucleotide sequence of the coding region of the wild-type gene corresponds to position 1264 in SEQ ID NO: 9.

The amino acid sequence of the Zwischenferment protein of strain DM658 (Zwf(A243T)) contains at position 243 the amino acid threonine (SEQ ID NO: 22). At the corresponding position of the wild-type protein is the amino acid alanine (SEQ ID NO: 10). Accordingly the allele is referred to as zwf(A243T). SEQ ID NO: 23 shows an internal segment of the coding sequence of the zwf(A243T) allele comprising the guanine adenine transition (see position 137 of SEQ ID NO: 23).

### Example 14

Transfer of the zwf allele zwf(A243T)

14.1 Isolation of a DNA Fragment Which Carries the zwf(A243T) allele

From strain DM658, chromosomal DNA is isolated by conventional methods (Eikmanns, et al., Microbiol. 140:1817-1828 (1994)). A DNA section which carries the zwf(A243T) allele with the base adenine at position 727 of the coding region (CDS) instead of the base guanine, which is at this position in the wild-type gene, is amplified with the aid of the polymerase chain reaction. On the basis of the sequence of the zwf gene of C. glutamicum, the following primer oligonucleotides are chosen for the polymerase chain reaction:

zwf_XL-A1:

(SEQ ID NO: 24)

5′ ga tct aga-agc tcg cct gaa gta gaa tc 3′
zwf_XL-E1:

(SEQ ID NO: 25)

5′ ga tct aga-gat tca cgc agt cga gtt ag 3′

The primers shown are synthesized by MWG Biotech (Ebersberg, Germany) and the PCR reaction is carried out by the standard PCR method of Innis, et al. (PCR Protocols. A Guide to Methods and Applications, Academic Press (1990)). The primers allow amplification of a DNA section approximately 1.75 kb in length which carries the zwf(A243T) allele (SEQ ID NO: 26). The primers moreover contain the sequence for a cleavage site of the restriction endonuclease XbaI, which is marked by underlining in the nucleotide sequence shown above.

The amplified DNA fragment of approximately 1.75 kb in length which carries the zwf(A243T) allele is cleaved with the restriction endonuclease XbaI, identified by electrophoresis in a 0.8% agarose gel and then isolated from the gel and purified by conventional methods (QIAquick Gel Extraction Kit, Qiagen, Hilden).

14.2 Construction of an Exchange Vector

The XbaI DNA fragment of approximately 1.75 kb length containing the zwf(A243T) allele (see Example 14.1) is incorporated into the chromosome of the C. glutamicum strain DSM5715 by means of replacement mutagenesis using the sacB system as described by Schaefer, et al. (Gene, 14:69-73 (1994)). This system allows for preparation and selection of allele exchanges occurring by homologous recombination.

The mobilizable cloning vector pK18mobsacB is digested with the restriction enzyme XbaI and the ends are dephosphorylated with alkaline phosphatase (Alkaline Phosphatase, Boehringer Mannheim, Germany). The vector prepared in this way is mixed with the zwf(A243T) fragment approx. 1.75 kb in size and the mixture is treated with T4 DNA ligase (Amersham-Pharmacia, Freiburg, Germany).

The E. coli strain S17-1 (Simon, et al., Bio/Technologie 1:784-791 (1993)) is then transformed with the ligation batch (Hanahan, In. DNA cloning. A Practical Approach. Vol. 1, ILR-Press, Cold Spring Harbor, N.Y., 1989). Selection of plasmid-carrying cells is made by plating out the transformation batch on LB agar (Sambrook; et al., Molecular Cloning: A Laboratory Manual, 2^{nd} ed., Cold Spring Harbor, N.Y., 1989), which was supplemented with 25 mg/l kanamycin.

Plasmid DNA is isolated from a transformant with the aid of the QIAprep Spin Miniprep Kit from Qiagen and checked by restriction cleavage with the enzyme PstI and subsequent agarose gel electrophoresis. The plasmid is called pK18mobsacB_zwf-(A243T) and is shown in FIG. 6.

14.3 Transfer of the allele

The vector pK18mobsacB_zwf(A243T) mentioned in Example 14.2 is transferred by conjugation by the protocol of Schaefer, et al. (J. Microbiol. 172:1663-1666 (1990)) into C. glutamicum strain DSM5715. The vector cannot replicate independently in DSM5715 and is retained in the cell only if it is integrated in the chromosome as the consequence of a recombination event. Selection for transconjugants, i.e., clones with integrated pK18mobsacB_zwf(A243T), is made by plating out the conjugation batch on LB agar (Sambrook, et al., Molecular Cloning: A Laboratory Manual, 2^{nd} ed., Cold Spring Harbor, N.Y., 1989), which is supplemented with 15 mg/l kanamycin and 50 mg/l nalidixic acid. Kanamycin-resistant transconjugants are plated out on LB agar plates containing 25 mg/l kanamycin and incubated for 24 hours at 33° C. A kanamycin-resistant transconjugant is called DSM5715::pK18mobsacB_zwf(A243T). As a result of the integration of plasmid vector pK18mobsacB_zwf(A243T) in the chromosome of strain DSM5715, the strain obtained, i.e. DSM5715::pK18mobsacB_zwf(A243T), contains the zwf wild type gene and the zwf(A243T) allele.

For selection of mutants in which excision of the plasmid has taken place as a consequence of a second recombination event, cells of the strain DSM5715::pK18 mobsacB_zwf(A243T) are cultured for 24 hours unselectively in LB liquid medium and then plated out on LB agar with 10% sucrose and incubated for 30 hours.

The plasmid pK18mobsacB_zwf(A243T), like the starting plasmid pK18mobsacB, contains, in addition to the kanamycin resistance gene, a copy of the sacB gene which codes for levan sucrase from Bacillus subtilis. The expression which can be induced by sucrose leads to the formation of levan sucrase, which catalyses the synthesis of the product levan, which is toxic to C. glutamicum. Only those clones in which the integrated plasmid pK18mobsacB_zwf(A243T) has excised as the consequence of a second recombination event therefore grow on LB agar containing sucrose. Depending on the position of the second recombination event with respect to the mutation site either allele exchange (i.e., incorporation of the mutation) occurs or the original copy (i.e. the wild type gene) remains in the chromosome of the host.

Approximately 40 to 50 colonies are tested for the phenotype “growth in the presence of sucrose” and “non-growth in the presence of kanamycin.” In 4 colonies which show the phenotype “growth in the presence of sucrose” and “non-growth in the presence of kanamycin,” a region of the zwf gene spanning the zwf(A243T) mutation is sequenced, starting from the sequencing primer zf_{—}1 (SEQ ID NO: 26), (prepared by GATC Biotech AG, Konstanz, Germany) to demonstrate that the mutation of the zwf(A243T) allele is present in the chromosome. The nucleotide sequence of primer zf_{—}1 is as follows:

zf_{—}1 (SEQ ID NO: 27): 5′ ggc tta eta ect gtc cat te 3′

A clone which contains the base adenine at position 727 of the coding region (CDS) of the zwf gene and thus has the zwf(A243T) allele in its chromosome was identified in this manner. This clone was called strain DSM5715zwf2_A243T.

Strain DSM5715zwf2_A243T was deposited at the Deutsche Sammlung fuer Mikroorganismen und Zellkulturen (DSMZ=German Collection of Microorganisms and Cell Cultures, Braunschweig, Germany) in accordance with the Budapest Treaty under DSM15237.

### EXAMPLE 15

Characterization and Determination of Glucose-6-Phosphate Dehydrogenase

15.1 Determination of the Glucose-6-Phosphate Dehydrogenase Activity of Strain DM658

For characterization of the activity of the glucose-6-phosphate dehydrogenase enzyme encoded by the zwf allele zwf(A243T), strain DM658 is incubated for 24 hours in LB media (Merck KG, Darmstadt, Germany). Culturing is carried out in a 25 ml volume in a 250 ml conical flask with baffles at 33° C. at 200 rpm on a shaking machine. For comparison, the wild-type strain ATCC13032 is incubated in parallel. The biomass is collected by centrifugation and subsequently washed in a Tris-HCl (100 mM) buffer at pH 7.8. The cells are solubilized using the Ribolyser system (Hybaid AG, Heidelberg, Germany). In this method, the cells are solubilized mechanically using 1.6 g glass beads (0.2 μm in diameter) and 0.6 g of a solution of Tris-HCl (100 mM)/NaCl buffer (520 mM) at pH 7.8. After centrifugation, the supernatant is isolated and used as crude extract. An aliquot of the supernatant is used for the determination of the total protein concentration using the colorimetric BCA method (Pierce, Rockford, Ill., USA, Order No. 23235ZZ). Another aliquot is used for the determination of the glucose-6-phosphate dehydrogenase activity.

Glucose-6-phosphate dehydrogenase (EC 1.1.1.49) catalyses the reaction: glucose-6-phosphate+NADP⁺→6-phosphoglucono-δ-lactone+NADPH. The assay system for determination of glucose-6-phosphate dehydrogenase activity contains 100 mM Tris-HCl (pH 7.8), 10 mM MgCl₂ and 260 μM NADP⁺. The reaction is initiated by addition of glucose-6-phosphate to give a final concentration of 7 mM glucose-6-phosphate. The absorption of NADPH is monitored at 340 nm with a Hitachi U3200 spectrophotometer (Nissei Sangyo, Duesseldorf, Germany) at 25° C.

For calculation of the volumetric enzyme activity in units per ml the following formula is used:

$$\frac{change of absorption of NADPH at 340 nm per minute}{6.22 * volume of crude extract used for the assay ( ml )}$$

To calculate the specific enzyme activity in Units per mg (U/mg; mU=milliunits/mg total protein) the enzyme activity is divided by the protein concentration of the crude extract.

Measurement of glucose-6-phosphate dehydrogenase activity in the presence of NADPH is done in an assay system containing 100 mM Tris-HCl (pH 7.8), 10 mM MgCl₂, 260 μM NADP⁺ and 260 μM NADPH. The reaction is initiated by the addition of glucose-6-phosphate to give a final concentration of 7 mM. The calculation of the enzyme activity in the presence of NADPH is done in the same way as described before. The results of this experiment are shown in Table 5.

TABLE 5

| | glucose-6-phosphate dehydrogenase | | |
|---|---|---|---|
| | activity in the absence | activity in the presence | residual |
| | of NADPH | of NADPH | activity |
| strain | (mU/mg protein) | (mU/mg protein) | (%) |
| ATCC13032 | 80 | 14 | 17.5 |
| DM658 | 130 | 84 | 64.6 |

15.2 Determination of the Glucose-6-Phosphate Dehydrogenase Activity of Strain DSM5715zwf2_A243T

For determination of the activity of the glucose-6-phosphate dehydrogenase enzyme encoded by the zwf allele zwf(A243T) contained in strain DSM5715zwf2_A243T the strain is incubated for 24 hours in LB media (Merck KG, Darmstadt, Germany). Culturing is carried out in a 25 ml volume in a 250 ml conical flask with baffles at 33° C. at 200 rpm on a shaking machine. For comparison the parental strain DSM5715 having a wild-type zwf gene is incubated in parallel. The preparation of the biomass is done as described in Example 15.1.

Measurement of the glucose-6-phosphate dehydrogenase activity in presence of its reaction end product NADPH is done in an assay system containing 100 mM Tris-HCl (pH 7.8), 10 mM MgCl₂, 260 μM NADP⁺, 7 mM glucose-6-phosphate and 400 μM NADPH. The enzyme activity in the presence of NADPH is calculated in the same way as described before. The results of this experiment are shown in Table 6.

TABLE 6

| | glucose-6-phosphate dehydrogenase | | |
|---|---|---|---|
| | activity in the | activity in the | |
| | absence | presence | residual |
| | of NADPH | of NADPH | activity |
| strain | (mU/mg protein) | (mU/mg protein) | (%) |
| DSM5715 | 86 | 13 | 15 |
| DSM5715zwf2_A243T | 64 | 18 | 28 |

### Example 16

Production of L-Lysine

The C. glutamicum strains DSM5715 and DSM5715zwf2_A243T, obtained in Example 14, are cultured in a nutrient medium suitable for the production of lysine and the lysine content in the culture supernatant is determined. For this, the strains are first incubated on an agar plate for 24 hours at 33° C. Starting from this agar plate culture, in each case a preculture is seeded (10 ml medium in a 100 ml conical flask). The medium MM is used as the medium for the precultures. The precultures are incubated for 24 hours at 33° C. at 240 rpm on a shaking machine. In each case a main culture is seeded from these precultures such that the initial OD (660 nm) of the main cultures is 0.1. The Medium MM is also used for the main cultures.

Medium MM

| | | |
|---|---|---|
| CSL | 5 | g/l |
| MOPS | 20 | g/l |
| Glucose (autoclaved separately) | 50 | g/l |
| Salts: | | |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL (corn steep liquor), MOPS (morpholinopropanesulfonic acid) and the salt solution are brought to pH 7 with aqueous ammonia and autoclaved. The sterile substrate and vitamin solutions, as well as the CaCO₃ autoclaved in the dry state, are then added. Culturing is carried out in a 10 ml volume in a 100 ml conical flask with baffles at 33° C. and 80% atmospheric humidity.

After 72 hours, the OD is determined at a measurement wavelength of 660 nm with a Biomek 1000 (Beckmann Instruments GmbH, Munich). The amount of lysine formed is determined with an amino acid analyzer from Eppendorf-BioTronik (Hamburg, Germany) by ion exchange chromatography and post-column derivation with ninhydrin detection. The result of the experiment is shown in Table 7.

TABLE 7

| | OD | Lysine HCl |
|---|---|---|
| Strain | (660 nm) | g/l |
| DSM5715 | 8.6 | 15.3 |
| DSM5715zwf2_A243T | 9.0 | 16.2 |

What is claimed is:

1. A process for the preparation of L-lysine by the fermentation of bacteria comprising the following steps:
a) fermenting L-lysine producing bacteria in which a zwf gene encoding the Zwischenferment protein is overexpressed relative to the wild-type bacteria;
b) concentrating L-lysine in the medium or in the cells of said coryneform bacteria; and
c) isolating the L-lysine produced;
wherein the intracellular activity of pyruvate oxidase encoded by the poxB gene is decreased or switched off.

2. The process of claim 1, wherein the endogenous zwf gene is used for overexpression.

3. The process of claim 1, wherein overexpression is achieved by transformation of bacteria with a vector.

4. The process of claim 3, wherein said vector comprises a zwf gene and a promoter.

5. The process of claim 1, wherein strains of the genus Corynebacterium are used.

6. A process for the preparation of L-amino acids selected from the group consisting of: L-threonine; L-isoleucine; and L-tryptophan; comprising the following steps:
a) fermenting L-amino acid producing bacteria in which a zwf gene encoding the Zwischenferment protein is overexpressed relative to the wild-type bacteria;
b) concentrating the L-amino acid in the medium or in the cells of the bacteria; and
c) isolating the L-lysine produced;
wherein the intracellular activity of the pyruvate oxidase encoded by the poxB gene is decreased or switched off.

7. A process for the preparation of L-lysine by fermentation of coryneform bacteria comprising the following steps:
a) fermenting L-lysine producing bacteria in which a zwf gene encoding the Zwischenferment protein is overexpressed relative to the wild-type bacteria;
b) concentrating the L-lysine in the medium or in the cells of the bacteria; and
c) isolating the L-lysine produced;
wherein the intracellular activity of the glucose 6-phosphate isomerase encoded by the pgi gene is decreased or switched off.

8. The process of claim 7, wherein the endogenous zwf gene is used for over-expression.

9. The process of claim 7, wherein overexpression is achieved by the transformation of bacteria with a plasmid vector carrying at least a zwf gene and a promoter.

10. The process of claim 7, wherein strains of the genus Corynebacterium are used.

11. A process for the preparation of L-amino acids selected from the group consisting of: L-threonine, L-isoleucine and L-tryptophan, by fermentation of bacteria comprising the following steps:
a) fermenting L-amino acid producing bacteria in which a zwf gene encoding the Zwischenferment protein is overexpressed relative to the wild-type bacteria;
b) concentrating the L-amino acid in the medium or in the cells of the bacteria; and
c) isolating the L-amino acid produced;
wherein the intracellular activity of the glucose 6-phosphate isomerase encoded by the pgi gene is decreased or switched off.

12. An L-amino acid producing coryneform microorganism, in which the intracellular activity of Zwischenferment is increased relative to the wild-type bacteria; and in which the intracellular activity of pyruvate oxidase is decreased or switched off.

13. An L-amino acid producing coryneform microorganism, in which the intracellular activity of Zwischenferment is increased and in which the intracellular activity of glucose 6-phosphate isomerase is decreased or switched off.

14. An isolated DNA consisting essentially of nucleotides 538 to 2079 of SEQ ID NO: 9.

15. A vector comprising the DNA of claim 14.

16. The plasmid vector pEC-TI 8mob2 deposited under the designation DSM13244 in E. coli K-12 DH5α and shown in FIG. 2.

17. A coryneform microorganism of the genus Corynebacterium, transformed by the introduction of the vector of either claim 15 or claim 16.

18. An isolated polynucleotide encoding a protein with the amino acid sequence of SEQ ID NO: 10, wherein at least one or more of the amino acids at positions 369 to 373 and/or one or more of the amino acids at positions 241 to 246 is exchanged by another proteinogenic amino acid.

19. An isolated polynucleotide encoding a protein with the amino acid sequence of SEQ ID NO: 10, wherein at least one or more of the amino acids selected from the group consisting of: L-arginine at position 370; L-valine at position 372; L-methionine at position 242; L-alanine at position 243; L-glutamic acid at position 244; and L-aspartic acid at position 245; is exchanged for any other proteinogenic amino acid.

20. An isolated polynucleotide encoding a protein selected from the group consisting of: a protein with the amino acid sequence of SEQ ID NO: 10, wherein at least L-alanine at position 243 is replaced with L-threonine; a protein with the amino acid sequence of SEQ ID NO: 10, wherein at least L-methionine at position 242 is replaced with L-leucine; a protein with the amino acid sequence of SEQ ID NO: 10, wherein at least L-methionine at position 242 is replaced with L-serine; a protein with the amino acid sequence of SEQ ID NO: 10, wherein at least L-aspartic acid at position 245 is replaced with L-serine, a protein with the amino acid sequence of SEQ ID NO: 10, wherein at least L-arginine at position 370 is replaced with L-methionine; and a protein with the amino acid sequence of SEQ ID NO: 10, wherein at least L-valine at position 372 is replaced with L-alanine.

21. An isolated polynucleotide encoding a protein comprising at least the amino acid sequence of SEQ ID NO: 22 amino acids 241 to 246 and optionally the amino acid sequence of SEQ ID NO: 10 amino acids 1 to 10.

22. An isolated polynucleotide consisting essentially of nucleotides 308 to 1849 of SEQ ID NO: 21.

23. The isolated polynucleotides of claims to 18 to 22, wherein said encoded protein has glucose 6-phosphate dehydrogenase activity.

24. The isolated polynucleotide of claim 23, encoding a protein that has glucose 6-phosphate dehydrogenase activity wherein said glucose 6-phosphate dehydrogenase activity is resistant to inhibition by NADPH.

25. A vector comprising the polynucleotide of any one of claims 18-22.

26. A coryneform microorganism of the genus Corynebacterium, transformed by the introduction of the vector of claim 25.

27. An isolated polynucleotide consisting essentially of the nucleotide sequence of SEQ ID NO: 21 and encoding a protein having glucose 6-phosphate dehydrogenase activity.

28. The isolated polynucleotide of claim 27 encoding a protein having glucose 6-phosphate dehydrogenase activity, wherein said protein comprises at least the N terminal sequence of SEQ ID NO: 10 amino acids 1 to 10.

29. A vector comprising the polynucleotide of either claim 27 or 28.

30. A bacterium comprising the isolated polynucleotide of any one of claims 18-22, 27 or 28.

31. The bacterium of claim 30, wherein said isolated polynucleotide is located in the chromosome of said bacterium.

32. The bacterium of claim 31, wherein said bacterium is a coryneform bacterium or Escherichia coli.

33. A bacterium comprising a polynucleotide encoding a protein with the amino acid sequence of SEQ ID NO: 10, wherein one or more of the amino acids at positions 369 to 373 and/or one or more of the amino acids at positions 241 to 246 is replaced by another proteinogenic amino acid.

34. An isolated bacterium comprising a polynucleotide encoding a protein with the amino acid sequence of SEQ ID NO: 10, wherein one or more of the amino acids selected from the group consisting of: L-arginine at position 370; L-valine at position 372; L-methionine at position 242; L-alanine at position 243; L-glutamic acid at position 244; and L-aspartic acid at position 245; is replaced with any other proteinogenic amino acid.

35. An isolated bacterium comprising a polynucleotide encoding a protein with the amino acid sequence of SEQ ID NO: 10, wherein at least L-alanine at position 243 is replaced with L-threonine.

36. An isolated bacterium comprising a polynucleotide encoding a protein, wherein said protein comprises at least the amino acid sequence of SEQ ID NO: 22 amino acids 241 to 246.

37. An isolated bacterium comprising a polynucleotide encoding a protein comprising at least the amino acid sequence of SEQ ID NO: 10 amino acids 1 to 10 and the amino acid sequence of SEQ ID NO: 22 amino acids 241 to 246.

38. An isolated bacterium comprising a polynucleotide with the nucleotide sequence of SEQ ID NO: 22 nucleotides 308 to 1849.

39. The isolated bacterium of claims 33 to 38 comprising a polynucleotide encoding a protein, wherein said protein has glucose 6-phosphate dehydrogenase activity.

40. The isolated bacterium of claim 39 comprising a polynucleotide encoding a protein having glucose 6-phosphate dehydrogenase activity, wherein said glucose 6-phosphate dehydrogenase activity is resistant to inhibition by NADPH.

41. The isolated bacterium of claims 33 to 38 comprising a polynucleotide encoding a protein, wherein the N terminal methionine is eliminated from said protein during processing within said bacterium.

42. The isolated bacterium of claim 33 to 38 wherein said bacterium is a coryneform bacterium.

43. Corynebacterium glutamicum DM658 deposited under DSM 7431.

44. Corynebacterium glutamicum DSM5715zwf2_A243T deposited under DSM14237.

45. A process for the preparation of an amino acid by the fermentation of an isolated coryneform bacterium comprising the following steps:
a) fermenting an amino acid producing bacterium comprising a polynucleotide encoding a protein with the amino acid sequence of SEQ ID NO: 10, wherein at least one or more of the amino acids at positions 369 to 373 and/or one or more of the amino acids at positions 241 to 246 is replaced by another proteinogenic amino acid, and
b) concentrating amino acid in the medium or in the cells of the bacterium.

46. The process of claim 45 step a), wherein said polynucleotide encodes a protein with the amino acid sequence of SEQ ID NO: 22.

47. The process of claim 45, wherein said amino acid is selected from the group consisting of L-lysine, L-threonine, L-isoleucine and L-tryptophan.

48. The process of claim 45 further comprising isolating said L-amino acid.

49. A process for the preparation of an amino acid by the fermentation of a coryneform bacterium, comprising the following steps:
a) fermenting the amino acid producing bacterium comprising an isolated polynucleotide encoding a protein with the amino acid sequence of SEQ ID NO: 10, wherein at least one or more of the amino acids at positions 369 to 373 and/or one or more of the amino acids at positions 241 to 246 is replaced by another proteinogenic amino acid, and
b) concentrating of the amino acid in the medium or in the cells of the bacterium.

50. The process of claim 49, step a), wherein said isolated polynucleotide encodes a protein with the amino acid sequence of SEQ ID NO: 22.

51. The process of claim 49, wherein said amino acid is selected from the group consisting of L-lysine, L-threonine, L-isoleucine and L-tryptophan.

52. The process of claim 49 further comprising isolating said L-amino acid.

53. A process for the preparation of an amino acid by fermentation of an isolated coryneform bacterium comprising the following steps:
a) fermenting an amino acid producing bacterium comprising a polynucleotide encoding a protein having glucose-6-phosphate dehydrogenase activity comprising at least the amino acid sequence of SEQ ID NO: 22 positions 241 to 246, and
b) concentrating of the amino acid in the medium or in the cells of the bacterium.

54. A process for the preparation of an amino acid by fermentation of a coryneform bacterium comprising the following steps:
a) fermenting an amino acid producing bacterium comprising an isolated polynucleotide encoding a protein having glucose-6-phosphate dehydrogenase activity with at least the amino acid sequence of SEQ ID NO: 22 positions 241 to 246, and
b) concentrating of the amino acid in the medium or in the cells of the bacterium.

55. The process of claims 53 or 54, wherein said amino acid is selected from the group consisting of L-lysine, L-threonine, L-isoleucine and L-tryptophan.

56. The process of claims 53 or 54 further comprising isolating said L-amino acid.

57. The process of claims 53 or 54 wherein said protein further comprises the N-terminal amino acid sequence of SEQ ID NO: 10 positions 1 to 10.
